SEQUENCE LISTING (90 sequences)

SEQ ID NO: 1 - protein, 24 aa, Homo sapiens
  PEPTIDE (1)...(24): alpha 1-24 fragment of fibrinogen E
ADSGEGDFLAEGGGVRGPRVVERH

SEQ ID NO: 2 - DNA, 72 bp, Homo sapiens
  CDS (1)...(72): coding sequece for alpha1-24 fragment of fibrinogen E
gcagatagtggtgaaggtgactttctagctgaaggaggaggcgtgcgtggcccaagggttgtggaaagacat

SEQ ID NO: 3 - protein, 9 aa, Homo sapiens
  PEPTIDE (1)...(9): anti-angiogenic peptide
XFLAEXXXV

SEQ ID NO: 4 - protein, 9 aa, Homo sapiens
  PEPTIDE (1)...(9): anti-angiogenic peptide
DXLAEXXXV

SEQ ID NO: 5 - protein, 9 aa, Homo sapiens
  PEPTIDE (1)...(9): anti-angiogenic peptide
DFXAEXXXV

SEQ ID NO: 6 - protein, 9 aa, Homo sapiens
  PEPTIDE (1)...(9): anti-angiogenic peptide
DFLXEXXXV

SEQ ID NO: 7 - protein, 9 aa, Homo sapiens
  PEPTIDE (1)...(9): anti-angiogenic peptide
DFLAXXXXV

SEQ ID NO: 8 - protein, 9 aa, Homo sapiens
  PEPTIDE (1)...(9): anti-angiogenic peptide
DFLAEXXXV

SEQ ID NO: 9 - protein, 9 aa, Hom sapiens
  PEPTIDE (1)...(9): anti-angiogenic peptide
DFLAEXXXX

SEQ ID NO: 10 - protein, 9 aa, Homo sapiens
  PEPTIDE (1)...(9): anti-angiogenic peptide
DFLAEGGGV

SEQ ID NO: 11 - protein, 10 aa, Homo sapiens
  PEPTIDE (1)...(10): anti-angiogenic peptide
GXXXXXXXXX

SEQ ID NO: 12 - protein, 11 aa, Homo sapiens
  PEPTIDE (1)...(11): anti-angiogenic peptide
EGXXXXXXXXX

SEQ ID NO: 13 - protein, 12 aa, Homo sapiens
  PEPTIDE (1)...(12): anti-angiogenic peptide
GEGXXXXXXXXX

SEQ ID NO: 14 - protein, 10 aa, Homo sapiens
  PEPTIDE (1)...(10): anti-angiogenic peptide
XXXXXXXXXR

SEQ ID NO: 15 - protein, 11 aa, Homo sapiens
  PEPTIDE (1)...(11): anti-angiogenic peptide
XXXXXXXXXRG

SEQ ID NO: 16 - protein, 12 aa, Homo sapiens
  PEPTIDE (1)...(12): anti-angiogenic peptide
XXXXXXXXXRGP

SEQ ID NO: 17 - protein, 20 aa, Homo sapiens
  PEPTIDE (1)...(20): anti-angiogenic peptide
XXXXXLXEXXGXXXPRVXXR

SEQ ID NO: 18 - protein, 21 aa, Homo sapiens
  PEPTIDE (1)...(21): anti-angiogenic peptide
SXXXXXLXEXXGXXXPRVXXR

SEQ ID NO: 19 - protein, 20 aa, Homo sapiens
  PEPTIDE (1)...(20): anti-angiogenic peptide
XXXXXLXEXXGXXXPRVVXR

SEQ ID NO: 20 - protein, 20 aa, Homo sapiens
  PEPTIDE (1)...(20): anti-angiogenic peptide
GEGXFLXEXXGXXXPRVVXR

SEQ ID NO: 21 - protein, 20 aa, Homo sapiens
  VARIANT (1)...(20): anti-angiogenic peptide
GEGXFLXXXXXXXXXXXXXX

SEQ ID NO: 22 - protein, 20 aa, Homo sapiens
  PEPTIDE (1)...(20): anti-angiogenic peptide
GEGDFLAEGGGVRGPRVVER

SEQ ID NO: 23 - protein, 20 aa, Homo sapiens
  PEPTIDE (1)...(20): anti-angiogenic peptide
GEGDFLAEGGGXRGPRVVER

SEQ ID NO: 24 - protein, 20 aa, Homo sapiens
  PEPTIDE (1)...(20): anti-angiogenic peptide
GEGDFLAEGGGVXGPRVVER

SEQ ID NO: 25 - protein, 20 aa, Homo sapiens
  PEPTIDE (1)...(20): anti-angiogenic peptide
GEGDFLAEGGGVRXPRVVER

SEQ ID NO: 26 - protein, 20 aa, Homo sapiens
  PEPTIDE (1)...(20): anti-angiogenic peptide
GEGDFLAEGGGVRGPRVXER

SEQ ID NO: 27 - protein, 20 aa, Homo sapiens
  PEPTIDE (1)...(20): anti-angiogenic peptide
GEGDFLAEGGGVRGPRVVXR

SEQ ID NO: 28 - protein, 20 aa, Homo sapiens
  PEPTIDE (1)...(20): anti-angiogenic peptide
GEGDFLAEGGGXXXPRVVXR

SEQ ID NO: 29 - protein, 20 aa, Homo sapiens
  PEPTIDE (1)...(20): anti-angiogenic peptide
GEGDFLAEGGGXXXPRVXXR

SEQ ID NO: 30 - protein, 11 aa, Homo sapiens
  PEPTIDE (1)...(11): anti-angiogenic peptide fragment
XFLAEGGGXRG

SEQ ID NO: 31 - protein, 24 aa, Homo sapiens
  PEPTIDE (1)...(24): anti-angiogenic peptide
ADSGEGDFLAEGGGVRGPRVVERH

SEQ ID NO: 32 - protein, 24 aa, Homo sapiens
  PEPTIDE (1)...(24): anti-angiogenic peptide
AASGEGDFLAEGGGVRGPRVVERH

SEQ ID NO: 33 - protein, 24 aa, Homo sapiens
  PEPTIDE (1)...(24): anti-angiogenic peptide
ADAGEGDFLAEGGGVRGPRVVERH

SEQ ID NO: 34 - protein, 24 aa, Homo sapiens
  PEPTIDE (1)...(24): anti-angiogenic peptide
ADSAEGDFLAEGGGVRGPRVVERH

SEQ ID NO: 35 - protein, 24 aa, Homo sapiens
  PEPTIDE (1)...(24): anti-angiogenic peptide
ADSGAGDFLAEGGGVRGPRVVERH

SEQ ID NO: 36 - protein, 24 aa, Homo sapiens
  PEPTIDE (1)...(24): anti-angiogenic peptide
ADSGEADFLAEGGGVRGPRVVERH

SEQ ID NO: 37 - protein, 24 aa, Homo sapiens
  PEPTIDE (1)...(24): anti-angiogenic peptide
ADSGEGAFLAEGGGVRGPRVVERH

SEQ ID NO: 38 - protein, 24 aa, Homo sapiens
  PEPTIDE (1)...(24): anti-angiogenic peptide
ADSGEGDALAEGGGVRGPRVVERH

SEQ ID NO: 39 - protein, 24 aa, Homo sapiens
  PEPTIDE (1)...(24): anti-angiogenic peptide
ADSGEGDFAAEGGGVRGPRVVERH

SEQ ID NO: 40 - protein, 24 aa, Homo sapiens
  PEPTIDE (1)...(24): anti-angiogenic peptide
ADSGEGDFLAAGGGVRGPRVVERH

SEQ ID NO: 41 - protein, 24 aa, Homo sapiens
  PEPTIDE (1)...(24): anti-angiogenic peptide
ADSGEGDFLAEAGGVRGPRVVERH

SEQ ID NO: 42 - protein, 24 aa, Homo sapiens
  PEPTIDE (1)...(24): anti-angiogenic peptide
ADSGEGDFLAEGAGVRGPRVVERH

SEQ ID NO: 43 - protein, 24 aa, Homo sapiens
  PEPTIDE (1)...(24): anti-angiogenic peptide
ADSGEGDFLAEGGAVRGPRVVERH

SEQ ID NO: 44 - protein, 24 aa, Homo sapiens
  PEPTIDE (1)...(24): anti-angiogenic peptide
ADSGEGDFLAEGGGARGPRVVERH

SEQ ID NO: 45 - protein, 24 aa, Homo sapiens
  PEPTIDE (1)...(24): anti-angiogenic peptide
ADSGEGDFLAEGGGVAGPRVVERH

SEQ ID NO: 46 - protein, 24 aa, Homo sapiens
  PEPTIDE (1)...(24): anti-angiogenic peptide
ADSGEGDFLAEGGGVRAPRVVERH

SEQ ID NO: 47 - protein, 24 aa, Homo sapiens
  PEPTIDE (1)...(24): anti-angiogenic peptide
ADSGEGDFLAEGGGVRGARVVERH

SEQ ID NO: 48 - protein, 24 aa, Homo sapiens
  PEPTIDE (1)...(24): anti-angiogenic peptide
ADSGEGDFLAEGGGVRGPAVVERH

SEQ ID NO: 49 - protein, 24 aa, Homo sapiens
  PEPTIDE (1)...(24): anti-angiogenic peptide
ADSGEGDFLAEGGGVRGPRAVERH

SEQ ID NO: 50 - protein, 24 aa, Homo sapiens
  PEPTIDE (1)...(24): anti-angiogenic peptide
ADSGEGDFLAEGGGVRGPRVAERH

SEQ ID NO: 51 - protein, 24 aa, Homo sapiens
  PEPTIDE (1)...(24): anti-angiogenic peptide
ADSGEGDFLAEGGGVRGPRVVERH

SEQ ID NO: 52 - protein, 24 aa, Homo sapiens
  PEPTIDE (1)...(24): anti-angiogenic peptide
ADSGEGDFLAEGGGVRGPRVVERH

SEQ ID NO: 53 - protein, 24 aa, Homo sapiens
  PEPTIDE (1)...(24): anti-angiogenic peptide
ADSGEGDFLAEGGGVRGPRVVERH

SEQ ID NO: 54 - protein, 24 aa, Homo sapiens
  PEPTIDE (1)...(24): anti-angiogenic peptide
ADSGEGDFLAEGGGVRGPRVVERH

SEQ ID NO: 55 - protein, 24 aa, Homo sapiens
  PEPTIDE (1)...(24): anti-angiogenic peptide
ADSGEGDFLAEGGGVRGPRVVERH

SEQ ID NO: 56 - protein, 15 aa, Homo sapiens
  PEPTIDE (1)...(15): anti-angiogenic peptide
ADSGEGDFLAEGGGV

SEQ ID NO: 57 - protein, 15 aa, Homo sapiens
  PEPTIDE (1)...(15): anti-angiogenic peptide
DSGEGDFLAEGGGVR

SEQ ID NO: 58 - protein, 15 aa, Homo sapiens
  PEPTIDE (1)...(15): anti-angiogenic peptide
SGEGDFLAEGGGVRG

SEQ ID NO: 59 - protein, 15 aa, Homo sapiens
  PEPTIDE (1)...(15): anti-angiogenic peptide
GEGDFLAEGGGVRGP

SEQ ID NO: 60 - protein, 15 aa, Homo sapiens
  PEPTIDE (1)...(15): anti-angiogenic peptide
EGDFLAEGGGVRGPR

SEQ ID NO: 61 - protein, 15 aa, Homo sapiens
  PEPTIDE (1)...(15): anti-angiogenic peptide
GDFLAEGGGVRGPRV

SEQ ID NO: 62 - protein, 15 aa, Homo sapiens
  PEPTIDE (1)...(15): anti-angiogenic peptide
DFLAEGGGVRGPRVV

SEQ ID NO: 63 - protein, 15 aa, Homo sapiens
  PEPTIDE (1)...(15): anti-angiogenic peptide
FLAEGGGVRGPRVVE

SEQ ID NO: 64 - protein, 15 aa, Homo sapiens
  PEPTIDE (1)...(15): anti-angiogenic peptide
LAEGGGVRGPRVVER

SEQ ID NO: 65 - protein, 15 aa, Homo sapiens
  PEPTIDE (1)...(15): anti-angiogenic peptide
AEGGGVRGPRVVERH

SEQ ID NO: 66 - protein, 16 aa, Homo sapiens
  PEPTIDE (1)...(16): anti-angiogenic peptide
ADSGEGDFLAEGGGVR

SEQ ID NO: 67 - protein, 28 aa, Homo sapiens
  PEPTIDE (1)...(28): anti-angiogenic peptide
ADSGEGDFLAEGGGVRGPRVVERHQSAC

SEQ ID NO: 68 - protein, 28 aa, Homo sapiens
  PEPTIDE (1)...(28): anti-angiogenic peptide
ADSGEGDFLAEGGGVRGPRVVERHQSAC

SEQ ID NO: 69 - protein, 23 aa, Homo sapiens
  PEPTIDE (1)...(23): anti-angiogenic peptide
DSGEGDFLAEGGGVRGPRVVERH

SEQ ID NO: 70 - protein, 22 aa, Homo sapiens
  PEPTIDE (1)...(22): anti-angiogenic peptide
SGEGDFLAEGGGVRGPRVVERH

SEQ ID NO: 71 - protein, 21 aa, Homo sapiens
  PEPTIDE (1)...(21): anti-angiogenic peptide
GEGDFLAEGGGVRGPRVVERH

SEQ ID NO: 72 - protein, 20 aa, Homo sapiens
  PEPTIDE (1)...(20): anti-angiogenic peptide
EGDFLAEGGGVRGPRVVERH

SEQ ID NO: 73 - protein, 19 aa, Homo sapiens
  PEPTIDE (1)...(19): anti-angiogenic peptide
GDFLAEGGGVRGPRVVERH

SEQ ID NO: 74 - protein, 18 aa, Homo sapiens
  PEPTIDE (1)...(18): anti-angiogenic peptide
DFLAEGGGVRGPRVVERH

SEQ ID NO: 75 - protein, 17 aa, Homo sapiens
  PEPTIDE (1)...(17): anti-angiogenic peptide
FLAEGGGVRGPRVVERH

SEQ ID NO: 76 - protein, 16 aa, Homo sapiens
  PEPTIDE (1)...(16): anti-angiogenic peptide
LAEGGGVRGPRVVERH

SEQ ID NO: 77 - protein, 15 aa, Homo sapiens
  PEPTIDE (1)...(15): anti-angiogenic peptide
AEGGGVRGPRVVERH

SEQ ID NO: 78 - protein, 14 aa, Homo sapiens
  PEPTIDE (1)...(14): anti-angiogenic peptide
EGGGVRGPRVVERH

SEQ ID NO: 79 - protein, 13 aa, Homo sapiens
  PEPTIDE (1)...(13): anti-angiogenic peptide
GGGVRGPRVVERH

SEQ ID NO: 80 - protein, 12 aa, Homo sapiens
  PEPTIDE (1)...(12): anti-angiogenic peptide
GGVRGPRVVERH

SEQ ID NO: 81 - protein, 10 aa, Homo sapiens
  PEPTIDE (1)...(10): anti-angiogenic peptide
FLAEGGGVRG

SEQ ID NO: 82 - protein, 11 aa, Homo sapiens
  PEPTIDE (1)...(11): anti-angiogenic peptide
DFLAEGGGVRG

SEQ ID NO: 83 - protein, 12 aa, Homo sapiens
  PEPTIDE (1)...(12): anti-angiogenic peptide
GDFLAEGGGVRG

SEQ ID NO: 84 - protein, 13 aa, Homo sapiens
  PEPTIDE (1)...(13): anti-angiogenic peptide
EGDFLAEGGGVRG

SEQ ID NO: 85 - protein, 14 aa, Homo sapiens
  PEPTIDE (1)...(14): anti-angiogenic peptide
GEGDFLAEGGGVRG

SEQ ID NO: 86 - protein, 10 aa, Homo sapiens
  PEPTIDE (1)...(10): anti-angiogenic peptide
GGVRGPRVVE

SEQ ID NO: 87 - protein, 11 aa, Homo sapiens
  PEPTIDE (1)...(11): anti-angiogenic peptide
GGGVRGPRVVE

SEQ ID NO: 88 - protein, 12 aa, Homo sapiens
  PEPTIDE (1)...(12): anti-angiogenic peptide
EGGGVRGPRVVE

SEQ ID NO: 89 - protein, 13 aa, Homo sapiens
  PEPTIDE (1)...(13): anti-angiogenic peptide
AEGGGVRGPRVVE

SEQ ID NO: 90 - protein, 14 aa, Homo sapiens
  PEPTIDE (1)...(14): anti-angiogenic peptide
LAEGGGVRGPRVVE

(19) United States
(12) Patent Application Publication
(10) Pub. No.: US 2005/0118598 A1
(43) Pub. Date: Jun. 2, 2005

(54) Organic compounds

(76) Inventors: Carlos Garcia-Echeverria, Basel (CH); Claire Lewis, Sheffield (GB); Jeffery Robinson, Surrey (GB)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/504,905

(22) PCT Filed: Feb. 19, 2003
(86) PCT No.: PCT/EP03/01698

(30) Foreign Application Priority Data
Feb. 19, 2002 (GB) 0203882.6
Feb. 19, 2002 (GB) 0203883.4
Jul. 23, 2002 (GB) 0217067.8

(51) Int. Cl. C12Q 1/68; C07K 14/47; C07K 7/08; C07K 7/06

(52) U.S. Cl. 435/6; 530/327; 530/328; 530/329; 530/330; 514/14; 514/15; 514/16; 514/17; 424/9.6; 435/69.1; 435/320.1; 435/325

(57) ABSTRACT

The invention relates to polypeptides and modified polypeptides derived from fibrinogen comprising one of the following sequences:
X₁FLAEX₆X₇X₈V
DX₂LAEX₆X₇X₈V
DFX₃AEX₆X₇X₈V
DFLX₄EX₆X₇X₈V
DFLAX₅X₆X₇X₈V
DFLAEX₆X₇X₈V
DFLAEX₆X₇X₈X₉ wherein X is any amino acid residue and which polypeptide has anti-angiogenic activity.

The invention relates to the anti-angiogenic polypeptides and modified polypeptides derived from fibrinogen including oligomers of said polypeptides.

Angiogenesis, the development of new blood vessels from an existing vascular bed, is a complex multistep process that involves the degradation of components of the extracellular matrix and then the migration, proliferation and differentiation of endothelial cells to form tubules and eventually new vessels. Angiogenesis is important in normal physiological processes including, by example and not by way of limitation, embryo implantation; embryogenesis and development; and wound healing. Excessive angiogenesis is also involved in pathological conditions such as tumour cell growth and non-cancerous conditions such as neovascular glaucoma, rheumatoid arthritis, psoriasis and diabetic retinopathy.

The vascular endothelium is normally quiescent. However, upon activation endothelial cells proliferate and migrate to form microtubules which will ultimately form a capillary bed to supply blood to developing tissues and, of course, a growing tumour. A number of growth factors have been identified which promote/activate endothelial cells to undergo angiogenesis. These include, by example and not by way of limitation; vascular endothelial growth factor (VEGF); transforming growth factor (TGFb); acidic and basic fibroblast growth factor (aFGF and bFGF); and platelet derived growth factor (PDGF) (1,2).

VEGF is a an endothelial cell-specific growth factor which has a very specific site of action, namely the promotion of endothelial cell proliferation, migration and differentiation. VEGF is a dimeric complex comprising two identical 23 kD polypeptides. The monomeric form of VEGF can exist as four distinct polypeptides of different molecular weight, each being derived from an alternatively spliced mRNA. Of the four monomeric forms, two exist as membrane bound VEGF and two are soluble. VEGF is expressed by a wide variety of cell/tissue types including embryonal tissues; proliferating keratinocytes; macrophages; tumour cells. Studies (2) have shown VEGF is highly expressed in many tumour cell-lines including glioma and AIDS-associated Kaposi's sarcoma. VEGF activity is mediated through VEGF specific receptors expressed by endothelial cells and tumour cells. Indeed, VEGF receptors are up-regulated in endothelial cells which infiltrate tumours thereby promoting tumour cell growth.

bFGF is a growth factor which functions to stimulate the proliferation of fibroblasts and endothelial cells. bFGF is a single polypeptide chain with a molecular weight of 16.5 Kd. Several molecular forms of bFGF have been discovered which differ in the length at their amino terminal region. However the biological function of the various molecular forms appears to be the same. bFGF is produced by the pituitary gland and is encoded by a single gene located on human chromosome 4.

A number of endogenous inhibitors of angiogenesis have been discovered, examples of which are angiostatin and endostatin, which are formed by the proteolytic cleavage of plasminogen and collagen XVIII respectively. Both of these factors have been shown to suppress the activity of pro-angiogenic growth factors such as vascular VEGF and bFGF. Both also suppress endothelial cell responses to VEGF and bFGF in vitro, and reduce the vascularisation and growth of experimental tumours in animal models.

Fibrinogen, the soluble circulating precursor of fibrin, is a dimeric molecule containing pairs of non-identical chains, (ie the α-, β- and γ-chains). These are arranged as three discrete domains, the two outer D-domains and the central E domain (4). Fibrinogen can be digested either by plasmin or thrombin.

The first step in plasmin cleavage of fibrinogen is the cleavage of the a chain C-terminal domain. Plasmin then cleaves the two D domains from the one E domain (consisting of the NH2 terminal regions of the α-, β- and γ-chains held together by disulphide bonds) and numerous smaller fragments including a small peptide, beta1-42 (amino terminal of the β-chain) (5). Thrombin, on the other hand, produces a fibrin monomer and two copies of fibrinopeptides A and B (4). Fibrinogen has been shown to accumulate around leaky blood vessels in solid tumours (5), Fibrinogen has also been shown to polymerise at host-tumour interface to form fibrin networks that promote tumour angiogenesis by supporting the adhesion, migration, proliferation and differentiation of endothelial cells (7).

The fibrin E-fragment (FnE-fragmert), produced by the proteolytic cleavage of fibrin, stimulates angiogenesis in the chorioallantoic membrane assay (8). Furthermore, the amount of this protein present in invasive breast carcinomas positively correlates with the degree of tumour vascularity (5).

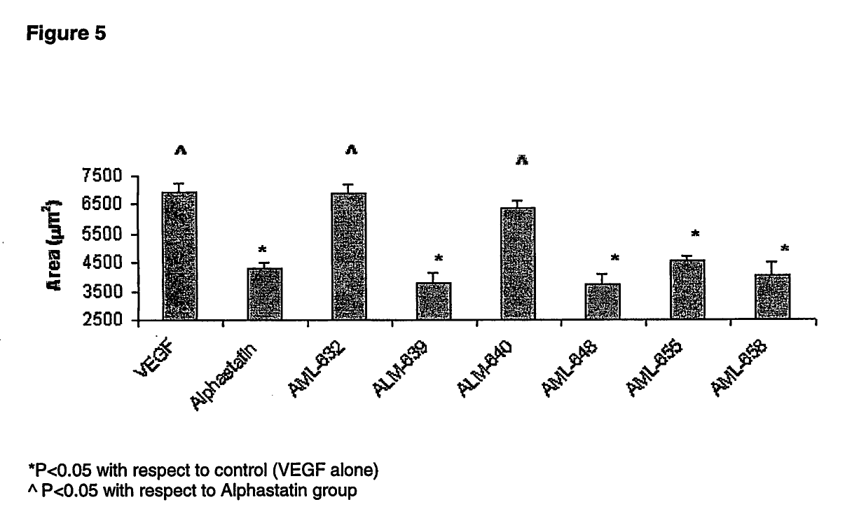

A potent, new inhibitor of angiogenesis, which is a 50 kDa proteolytic fragment of fibrinogen, fibrinogen E, is disclosed in our co-pending published application, WO01/88129. We have also identified a domain within the fibrinogen E fragment which has the same anti-angiogenic activity as the very much larger fibrinogen E fragment. The domain is located at the amino terminus of the α chain and is referred to as α1-24 and is disclosed in our co-pending application WO02/18440. Peptides and peptide variants derived from the domain have anti-angiogenic activity. The content of WO02/18440, with respect to DNA sequence disclosed in FIG. 5A and the peptides and peptide variants disclosed in FIG. 5B and on page 3, 4 and 5 we hereby disclaim.

Surprisingly, we have now found, in accordance with the present invention, that a fragment comprising a short sequence motif of the α1-24 peptide is sufficient for said anti-angiogenic activity. Accordingly, the invention provides in a first aspect a polypeptide of 15 or less amino acid residues comprising one of the following sequences:

X₁FLAEX₆X₇X₈V
DX₂LAEX₆X₇X₈V
DFX₃AEX₆X₇X₈V
DFLX₄EX₆X₇X₈V
DFLAX₅X₆X₇X₈V
DFLAEX₆X₇X₈V
DFLAEX₆X₇X₈X₉

wherein X is any amino acid residue and which polypeptide has anti-angiogenic activity. As used herein, X with a subscript number refers to a variable amino acid at a specific position, whereas X without a subscript number refers to a variable amino acid residue independent of its position and may refer to any of the X with subscript number. X may be a proteinogenic or a non-proteinogenic amino acid.

As further described hereinbelow, amino acid residues may be replaced without substantially reducing anti-angiogenic activity. Preferably, at least 90% of the anti-angiogenic activity is retained, more preferably at least 80%, or at least 70%, or at least 60%, or at least 50% is retained.

In a preferred embodiment of the present invention, X₁ is an acidic amino acid, X₂, X₃, X₄, X₅ are selected from the group consisting of glycine, alanine, valine, leucine, isoleucine, proline, X₆, X₇, X₈ may be any amino acid, may be a proteinogenic or a non-proteinogenic amino acid, and X₉ is a non-polar amino acid. In a particularly preferred embodiment, said polypeptide comprises the sequence DFLAEGGGV.

As will be apparent to one skilled in the art and as further described hereinbelow, peptides can be modified to improve pharmacological properties by incorporation of “non-proteinogenic” amino-acids without having a deteriorating effect on the biological activity of said peptide. Accordingly, in a preferred embodiment of the present invention, one or both of amino acid residues X₁ and X₅ are gamma-carboxy-L-glutamic acid, L-2-aminoadipic acid, L-3-aminoadipic acid or (+/−)-lminocyclopentane-cis-1,3-dicarboxylic acid. In addition or alternatively, X₂ is an amino acid residue selected from the group consisting of L-2-naphthylalanine, L-1,2,3,4-tetrahydroisoquinoline-3-carboxylic acid and 4,4′-biphenylalanine. In addition or alternatively, one or more of amino acid residues X₃, X₄ or X₉ are selected from the group consisting of 1-aminocyclopropanecarboxylic acid, 3-aminopentane-3-carboxylic acid, R-2-amino-2-cyclohexyl-propanoic acid.

In another embodiment of the above aspect, the present invention provides a polypeptide of 15 or less amino acid residues comprising a sequence selected from the group consisting of:

X₁X₂X₃X₄X₅X₆X₇X₈X₉R
X₁X₂X₃X₄X₅X₆X₇X₈X₉RG
X₁X₂X₃X₄X₅X₆X₇X₈X₉RGP

wherein X₁, X₂, X₃, X₄, X₅, X₆, X₇, X₈ and X₉, respectively, are defined as above.

In another embodiment of the above aspect, the present invention provides a polypeptide of 15 or less amino acid residues comprising a sequence selected from the group consisting of:

GX₁X₂X₃X₄X₅X₆X₇X₈X₉
EGX₁X₂X₃X₄X₅X₆X₇X₈X₉
GEGX₁X₂X₃X₄X₅X₆X₇X₈X₉

wherein X₁, X₂, X₃, X₄, X₅, X₆, X₇, X₈ and X₉, respectively, are defined as above.

In accordance with another aspect of the present invention, it was found that one or more of amino acid residues X₇, X₈ and X₉ can be replaced with a flexible chemical linker without without substantially reducing anti-angiogenic activity. Accordingly, the present invention provides a polypeptide, said polypeptide having one or more of amino acid residues X₇, X₈ and X₉ replaced by a flexible chemical linker. Preferably, the linker contains a “backbone” with 3 to 40, preferably 4 to 30, more preferably 5 to 15, most preferably 6 to 12 atoms, linking the flanking amino acids. The backbone atoms are preferably C-atoms, but may also contain heteroatoms such as O, N, P or S. The backbone may further be substituted with alkyl-, aryl- or alkoxy-groups or halogen. Flexible chemical linkers include by examples and not by way of limitation: —CH₂—CH₂—CH₂)ₓ—, —(CR₁R₂—CR₃R₄)ₓ—, —CH₂CH₂O)ₓ—, —(NH—CR₁R₂—CR₃R₄—CO)ₓ— wherein x=1-10 and wherein R₁, R₂, R₃, R₄, R₅ and R₆ are for instance a hydrogen-halogen-, alkyl-, aryl-, arlylalkyl- or alkoxy-radical, may be the same or different, and may further contain heteroatoms. Preferred chemical linkers comprise a backbone of one ore more ethyleneglycol or beta-amino acid, or gamma-amino acid moieties. Also cyclic linkers may be used such as. Examples for cyclic linkers include without limitation 3-amino-4-pyrazolecarboxylic acid, 4-amino-5-carboxy-2-hydroxypyrimidine, anthranilic acid derivatives, 3-amino-1-indanecarboxylic acid.

In accordance with a preferred embodiment of the above aspects, the polypeptide has a length of 9, 10, 11, 12, 13, or 14 amino acid residues. More preferred is a length of 9 or 10 amino acid residues.

In accordance with another preferred embodiment of the above aspects, said polypeptides consist of said amino acids.

Reference to anti-angiogenic activity is determined by assays herein disclosed. For example, the polypeptides of the invention are tested by in vitro assays which include the inhibition of endothelial cell mediated tubule formation, inhibition of endothelial cell migration, inhibition of VEGF and bFGF induced endothelial cell proliferation and endothelial cell cytotoxicity assays. In a preferred embodiment, the anti-angiogenic compounds of the present invention inhibit tubule formation, cell migration or cell proliferation by at least 10 percent, preferably at least 20 percent, more preferably at least 30 percent and most preferably at least 50 percent relative to a control without said polypeptide in an assay as herein described. In a more preferred embodiment, the assay is a proliferation assay according to the Example hereinbelow.

Polypeptides can also be tested in vivo using murine tumour models as herein disclosed. In a preferred embodiment, the anti-angiogenic compounds of the present invention inhibit tumour growth by at least 10 percent, preferably at least 20 percent, more preferably at least 30 percent and most preferably at least 50 percent relative relative to a control without said polypeptide.

A variant, i.e. a fragment polypeptide and reference polypeptide may differ in amino acid sequence by one or more substitutions, additions, deletions, truncations which may be present in any combination. Among preferred variants are those that vary from a reference polypeptide by conservative amino acid substitutions. Such substitutions are those that substitute a given amino acid by another amino acid of like characteristics. The following non-limiting list of amino acids are considered conservative replacements (similar): a) serine, and threonine; b) glutamic acid and asparatic acid; c) asparagine and glutamine d) arginine and lysine; e) isoleucine, leucine, methionine and valine and f) phenylalanine, histidine, tyrosine and tryptophan. Most highly preferred are variants which retain the same biological function and activity as the reference polypeptide from which it varies.

A functionally equivalent polypeptide according to the invention is a variant wherein one or more amino acid residues are substituted with conserved or non-conserved amino acid residues, or one in which one or more amino acid residues includes a substituenti group. Conservative substitutions are the replacements, one for another, among the aliphatic amino acids Ala, Val, Leu and Ile; interchange of the hydroxl residues Ser and Thr; exchange of the acidic residues Asp and Glu; substitution between amide residues Asn and Gln; exchange of the basic residues Lys and Arg; and replacements among aromatic residues Phe, His, Tyr and Trp.

In addition, the invention features polypeptide sequences having at least 75% identity with the polypeptide sequences as herein disclosed, or fragments and functionally equivalent polypeptides thereof. In one embodiment, the polypeptides have at least 85% identity, more preferably at least 90% identity, even more preferably at least 95% identity, still more preferably at least 97% identity, and most preferably at least 99% identity with the amino acid sequences illustrated herein.

The polypeptides of the present invention are preferably composed of alpha-amino acid residues. The invention, however, is not limited to polypeptides composed of alpha-amino acid residues, polypeptides comprising other types of residues, such as beta- or gamma-amino acid residues are also contemplated by the present invention. Any type of suitable residues may be used in accordance with the present invention so long as the polypeptide does not lose its ability to adopt the required biologically active three dimensional conformation.

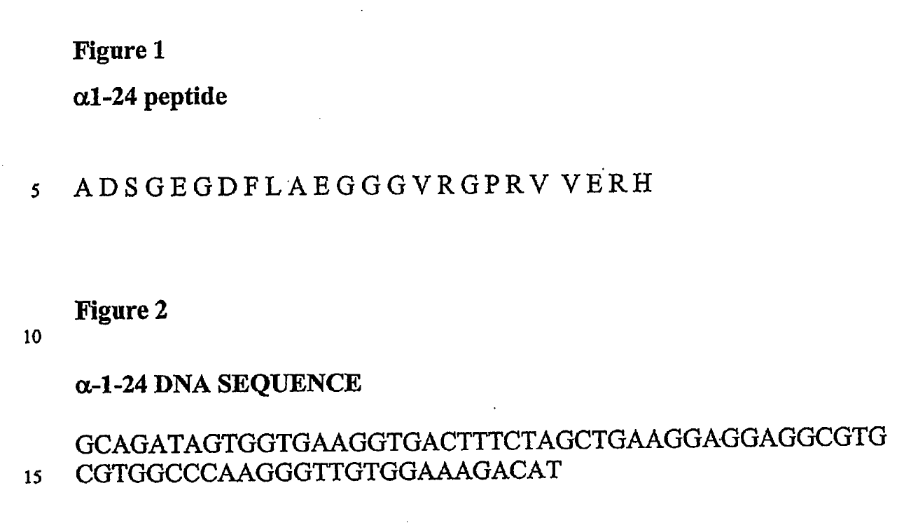

According to one aspect to the invention there is provided a polypeptide, or part thereof, comprising an amino acid sequence as represented by the sequence presented in FIG. 1 which has been modified by deletion, addition or substitution of at least one amino acid residue wherein said modified polypeptide has anti-angiogenic activity.

According to another aspect of the invention there is provided a polypeptide comprising an amino acid sequence selected from the group consisting of:

i) a peptide of the sequence, or part thereof:

XXXXXLXEXXGXXXPRVXXR

wherein X is any amino acid residue;
ii) a peptide as represented in (i) wherein amino acid residue X is selected from the following group: alanine, valine, leucine, isoleucine, proline; and
iii) a peptide represented in (i) or (ii) which has anti-angiogenic activity.

In a preferred embodiment of the invention said polypeptide comprises an amino acid sequence, or part thereof, consisting of the sequence:

SXXXXXLXEXXGXXXPRVXXR

In a preferred embodiment of the invention said polypeptide comprises an amino acid sequence as represented by the sequence:

XXXXXLXEXXGXXXPRVVXR

In a yet further preferred embodiment of the invention said polypeptide comprises an amino acid sequence as represented by the sequence:

GEGXFLXEXXGXXXPRVVXR

In a yet further preferred embodiment of the invention said polypeptide comprises an amino acid sequence as represented by the sequence:

GEG XFL XXX XXXXX XXXX XX.

In a preferred embodiment of the invention said polypeptide comprises an amino acid sequence as represented by the sequences presented in table 1. Preferably said polypeptides comprising said sequence have anti-angiogenic activity.

In a yet further preferred embodiment of the invention said polypeptide comprises an amino acid sequence selected from the group consisting of:

GEG DFL AEG GGV RGP RVVE R
GEG DFL AEG GGX RGP RVVE R
GEG DFL AEG GGV XGP RVVE R
GEG DFL AEG GGV RXP RVVE R
GEG DFL AEG GGV RGP RVXE R
GEG DFL AEG GGV RGP RVVXR
GEG DFL AEG GGXXXP RVVX R
GEG DFL AEG GGXXXP RVXXR.

X is any amino acid residue or, preferably, X is selected from the group consisting of alanine, valine, leucine, isoleucine, or proline. More preferably X is alanine.

In yet a further preferred embodiment of the invention the peptide comprises an amino acid sequence as represented by the overlapping part of two fragments presented in table 1. In a more preferred embodiment, said peptide is derived from the overlapping part of the peptides AHI-401 and AHI-378 in table 1. Preferably, said peptide derived from the overlapping part of the peptides AHI-401 and AHI-378 comprises one additional amino acid residue at the N-terminus.

Thus, in a preferred method of the invention said peptide comprises an amino acid sequence as represented by the sequence:

XFLAEGGGVXG

X is any amino acid residue selected from the group consisting of A, R, N, D, C, E, Q, G, H, I, L, K, M, F, P, S, T, W, Y, V. Preferably X is selected from the group consisting of A, V, L, I and P, more preferably X is a basic amino acid selected from the group consisting of H, R and K, or an acidic amino acid selected from the group consisting of D and E.

In another preferred embodiment, the N-terminal X is selected from the group consisting of D and E, whereas the C-terminal X is selected from the group consisting of H, R and K, or alternatively, the N-terminal amino acid is selected from the group consisting of H, R and K, whereas the C-terminal X is selected from the group consisting of D and E. In a particularly preferred embodiment, the N-terminal X is D and the C-terminal X is R.

In another preferred embodiment, the C-terminal X is selected from the group consisting of L-2,4-diaminobutyric acid, R-aminocarnitine, L-alpha-amino-gamma-guanidinobutyric acid or ornithine.

In a further preferred embodiment of the invention said part thereof is represented by the amino acid sequence from+1 to+15 of the amino acid sequence:

SXXXXXLXEXXGXXXPRVXXR

In a further preferred embodiment of the invention said part thereof is represented by the amino acid sequence from+6 to+21 of the amino acid sequence:

XXXXXLXEXXGXXXPRVXXR

In a yet further preferred embodiment of the invention said part thereof is represented by the amino acid sequence+6 to+15 of the amino acid sequence

XXXXXLXEXXGXXXPRVXXR

In a further preferred embodiment of the invention said polypeptide consists of the peptide amino acid sequences as herein disclosed.

In a further preferred embodiment of the invention said polypeptide is 10, 11, 12, 13, 14, 15, 16, 17, 18, 19, 20, 21, 22, or 23 amino acid residues in length and comprises an amino acid sequence according to the invention.

It will be apparent to one skilled in the art that modification to the amino acid sequence of polypeptides according to the invention could enhance the binding and/or stability of the polypeptide with respect to its target sequence. In addition, modification of the polypeptide may also increase pharmacological properties of the polypeptide, such as for example the in vivo stability, thereby reducing the effective amount of polypeptide necessary to inhibit angiogenesis. This would advantageously reduce undesirable side effects which may result in vivo.

Numerous peptide-modifications suitable for enhancing the pharmacological properties of the polypetide are known in the art. For example, the peptide backbone can be chemically modified, for instance as described in Gilles Guichard, Solid-phase synthesis of pseudopeptides and oligomeric peptide backbone mimetics, in Solid-phase synthesis—A practical guide,; edited by Steven A. Kates or in Fernando Albericio, Marcell Dekker, New York—Basel, pp 649-703, 2000, the contents of which are herewith incorporated by reference. Examples of backbone modifications include without limitation: beta-peptides, depsipeptides, oligosulfonamides, oliogureas and thioureas, oligocarbamates, peptoids, and azapeptides.

Another example of a suitable modification is the cyclisation of a peptide according to the invention. Said cyclisation may be head to tail cyclisation, side-chain to side-chain cyclisation, side-chain to end cyclisation, branched cyclisation, or backbone to backbone cyclisation. Cyclisation of peptides are for instance described in the following references, the contents of which are herewith incorporated by reference: Paolo Rovero, Homodetic cyclic peptides, in Solid-phase synthesis—A practical guide,; edited by Steven A. Kates and Fernando Albericio, Marcell Dekker, New York—Basel, pp 331-364, 2000. S A Kates, N A Sole, F Albericio, G. Barany, in C. Basava, G M Anantharamaiah, eds. Peptides: design, synthesis, and biological activity. Boston, Birkhauser, 1994, pp 39-58.

Other suitable modifications include the incorporation of “non-protein amino acids”.

As used herein, the term “amino acid” or “proteinogenic amino acid” refer to an amino acid which is a natural building block of a protein, i.e. an amino acid which is incorporated into a protein in the process of translating the genetic code into a protein. The interchangeably used terms “non-protein amino acid”, “non-proteinogenic amino acid” or “non-coded amino acid” include any compound with a chemical structure similar to an amino acid, but not naturally used in the process of translating the genetic code into a protein.

Accordingly, proteinogenic amino acids may for instance be replaced by the alpha-methyl derivative or by the alpha-ethyl derivative. In a preferred embodiment the peptide comprises an alphamethyl derivative selected from the group consisting of: alpha-methyl-L-alanine, alpha-methyl-L-arginine, alpha-methyl-L-asparagine, alpha-methyl-L-aspartic acid, alpha-methyl-Lcysteine, alpha-methyl-L-glutamatic acid, alpha-methyl-L-glutamine, alpha-methyl-L-glycine, alpha-methyl-L-histidine, alpha-methyl-L-isoleucine, alpha-methyl-L-leucine, alpha-methyl-L-lysine, alpha-methyl-L-methionine, alpha-methyl-L-phenylalanine, alpha-methyl-L-proline, alpha-methyl-L-serine, alpha-methyl-1-threonine, alpha-methyl-L-tryptophan, alpha-methyl-L-tyrosine and alpha-methyl-L-valine.

Another example of a suitable modification may be the replacement of amino acids for intstance by the N-methyl derivative or the N-ethyl derivative. In another preferred embodiment the peptide comprises an N-methyl derivative selected from the group consisting of: N-methyl-L-alanine, N-methyl-L-arginine, N-methyl-L-asparagine, N-methyl-L-aspartic acid, N-methyl-L-cysteine, N-methyl-L-glutamic acid, N-methyl-L-glutamine, N-methyl-L-glycine, N-methyl-L-histidine, N-methyl-L-isoleucine, N-methyl-L-leucine, N-methyl-L-lysine, N-methyl-L-methionine, N-methyl-L-phenylalanine, N-methyl-L-proline, N-methyl-L-serine, N-methyl-L-threonine, N-methyl-L-tryptophan, N-methyl-L-tyrosine and N-methyl-L-valine.

As will be apparent to the person of skill in the art, groups of amino acids with similar properties (for instance hydrophobic amino acids) are preferably replaced by “non-protein” amino acids with similar properties, analogous to what is known in the art as conservative amino acid substitutions. As used herein, “conservative” amino acids include both proteinogenic and non-proteinogenic amino acids with similar properties. Thus, a hydrophobic proteinogenic amino acid may be exchanged by a hydrophobic non-proteinogenic amino acid, a polar proteinogenic amino acids may be exchanged by a polar non-proteinogenic amino acids etc.

Accordingly, hydrophobic amino acid may be replaced by alpha,alpha-disubstituted linear or cylic amino acid. Without intending to be bound to these examples, hydrophobic amino acids such as glycine, alanine, leucine, isoleucine, valine, phenylalanine or proline may be replaced by 1-aminocyclopropanecarboxylic acid, 3-aminopentane-3-carboxylic acid, R-2-amino-2-cyclohexyl-propanoic acid; acidic amino acids such as glutamic acid or aspartic acid may be replaced by gamma-carboxy-L-glutamic acid, L-2-aminoadipic acid, L-3-aminoadipic acid or (+/−)-lminocyclopentane-cis-1,3-dicarboxylic acid acid. Further examples include, Cha, O-NaI, Aib (aminoisobutyric acid), Acₙc (α,α-disubstituted cyclic α-amino acid, n from 3 to 7, both inclusived; n refers to the number of carbons in the ring), Abu (2-aminobutyric acid), Nle, Nva (norvaline), Bpa (p-benzoyl-phenylalanine), hphe (homo-Phe), hPro (homo-Pro), 1-Nal(p-(1-naphthyl)alanine), 2-Nal(3-(2-naphthyl)alanine), Oic (octahydroindode-2-carboxylic acid), Tic (1,2,3,4-tetrahydroisoquinoline-3-carboxylic acid), Pen (penicillamine), Phg (phenylglycine), Tle (tert-leucine), p-X-Phe (X=Br, F, I, Cl, phenyl, CN, NO₂), Thi α-(2-thienyl)-alanine), and their homologues.

Furthermore, single proteinogenic amino acids may also be replaced by structurally similar “non-protein” amino acids derivatives. Structurally similar derivatives for a given proteinogenic amino acid are known in the art. For instance, arginine may be replaced by L-2,4-diaminobutyric acid, R-aminocarnitine, L-alpha-amino-gamma-guanidinobutyric acid or ornithine; phenylalanine may be replaced by L-2-naphthylalanine, L-1,2,34-tetrahydroisoquinoline-3-carboxylic acid or 4,4-biphenylalanine, p-benzoyl-phenylalanine, hphe; proline by 3-hydroxyproline, 4-hydroxyproline; lysine by 5-hydroxylysine, allo-hydroxylysine, N⁶-acetyllysine, N⁶-methyllysine, N⁶,N⁶-dimethyllysine, N⁶,N⁶,-trimethyllysine; alanine by cyclohexyalanine. Other modifications include amino acids with a C₂, C₃ or C₄ alkyl R group optionally substituted by 1, 2 or 3 substituents selected from halo (eg F, Br, I), hydroxy or C₁-C₄ alkoxy.

Another suitable modification of the peptides of the invention is the replacement of one or more L-amino acids may by the corresponding D-amino acid. Both, the proteinogenic or “non-protein” L-amino acids may be replaced by the corresponding D-amino acids.

A further suitable peptide derivative in accordance with the present invention is a retro-inverso peptidomimetic as described in the following references, the content of which herewith is incorporated by reference: M. Goodman, M. Chorev Acc Chem Res 12, 1-7, 1979, M. Chorev, M. Goodman Acc Chem Res 26, 266-273, 1993 and M. Chorev, M. Goodman Trend Biotechnol 13, 438-445, 1995.

In another embodiment of the present invention, the C-terminus is modified. C-terminal modifications include, by examples and not by way of limitation: carboxylic acid, esters, carboxamides, or alcohol. In a preferred embodiment, the C-terminal modification is a methyl-, ethyl-, propyl-, butyl- or isopropyl-ester. In another preferred embodiment, the C-terminal modification is CONH₂, CONHCH₃ (methyl amide) or CON(CH₃)₂ (dimethylamide). In another preferred embodiment, the C-terminal modification is CH₂OH.

In further embodiment of the present invention, the N-terminus is modified. N-terminal modifications include, by example and not by way of limitation, carboxamide, carbamate, urea, sulphonamide, acetylation and alkylation.

In a further preferred embodiment of the invention said polypeptide, is modified at both C-terminus and N-terminus.

Alternatively or preferably, said modification includes the use of modified amino acids in the production of recombinant or synthetic forms of polypeptides according to the invention.

In a further preferred embodiment of the invention there is provided a polypeptide according to the invention which polypeptide comprises at least one modified amino acid wherein X denotes the position of said modified amino acid.

The incorporation of modified amino acids may confer advantageous properties on polypeptides according to the invention. For example, the incorporation of modified amino acids may increase the affinity of the polypeptide for its binding site, or the modified amino acids may confer increased in vivo stability on the polypeptide thus allowing a decrease in the effective amount of therapeutic polypeptide administered to a patient.

It will also be apparent to one skilled in the art that fragments of polypeptides which retain anti-angiogenic activity could be recovered by fractionation of the intact polypeptide using, for example, proteolytic enzymes. Alternatively, fragments could be synthesised de novo and also modified by, for example, cyclisation. Cyclisation is known in the art, (see Scott et al Chem Biol (2001), 8:801-815; Gellerman et al J. Peptide Res (2001), 57: 277-291; Dutta et al J. Peptide Res (2000), 8:398-412; Ngoka and Gross J Amer Soc Mass Spec (1999), 10:360-363.

In a preferred embodiment of the invention the polypeptides according to the invention are modified by cyclisation.

According to a further aspect the invention there is provided a pharmaceutical composition comprising a polypeptide, or part thereof, according to the invention.

According to a further aspect of the invention there is provided an pharmaceutical composition comprising two or more polypeptides according to the invention wherein said pharmaceutical composition has anti-angiogenic activity.

In a preferred embodiment of the invention said two or more polypeptides are linked by a linker molecule. Preferably said linker molecule is a flexible linker.

In a further preferred embodiment of the invention said pharmaceutical composition comprises a plurality of polypeptides according to the invention. Preferably said pharmaceutical composition has 3, 4, 5, 6, 7, 8, 9, or 10 polypeptides linked together as an oligomeric polypeptide. Preferably said polypeptide has greater than 10 polypeptides according to the invention.

In a further preferred embodiment of the invention said pharmaceutical composition is a dimer of two polypeptides according to the invention.

In a further preferred embodiment of the invention said linker is a polypeptide linking molecule. In a preferred embodiment of the invention said polypeptide linking molecule comprises at least one amino acid residue which links at least two polypeptides according to the invention.

In a further preferred embodiment of the invention said polypeptide linking molecule comprises at least 2, 3, 4, 5, 6, 7, 8, 9, or 10 amino acid residues. In a further embodiment of the invention said linking molecule comprises more than 10 amino acid residues.

In an alternative embodiment of the invention, the polypeptide is a fusion protein comprising an inframe translational fusion of the polypeptides according to the invention.

It will be apparent that the invention encompasses the formation oligomeric polypeptides which comprise identical polypeptides according to the invention, herein referred to as homo-oligomeric as well as polypeptides comprising different modified polypeptides, referred to as hetero-oligomeric.

It will be apparent to one skilled in the art that alternative linkers can be used to link polypeptides, for example the use of chemical protein crosslinkers. For example homo-bifunctional crosslinker such as disuccinimidyl-suberimidate-dihydrochloride; dimethyl-adipimidate-dihydrochloride; 1,5,-2,4 dinitrobenezene or hetero-bifunctional crosslinkers such as N-hydroxysuccinimidyl 2,3-dibromopropionate; 1-ethyl-3-[3-dimethylaminopropyl] carbodiimide hydrochloride; succinimidyl 4-[n-maleimidomethyl]-cyclohexane-1-carboxylate.

When administered, the pharmaceutical compositions of the present invention are administered in pharmaceutically acceptable preparations. Such preparations may routinely contain pharmaceutically acceptable concentrations of salt, buffering agents, preservatives, compatible carriers, supplementary immune potentiating agents such as adjuvants and cytokines and optionally other therapeutic agents, such as chemotherapeutic agents.

The pharmaceutical compositions of the invention can be administered by any conventional route, including injection or by gradual infusion over time. The administration may, for example, be oral, intravenous, intraperitoneal, intramuscular, intracavity, subcutaneous, or transdermal.

The compositions of the invention are administered in effective amounts. An “effective amount” is that amount of a composition that alone, or together with further doses, produces the desired response. In the case of treating a particular disease, such as cancer, the desired response is inhibiting the progression of the disease. This may involve slowing the progression of the disease temporarily, although more preferably, it involves halting the progression of the disease permanently. This can be monitored by routine methods.

Such amounts will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation.

The pharmaceutical compositions used in the foregoing methods of treatment preferably are sterile and contain an effective amount of polypeptide, oligomeric agent or nucleic acid encoding said polypeptide or oligomeric agent, for producing the desired response in a unit of weight or volume suitable for administration to a patient.

The doses of polypeptide/oligomer, or nucleic acid encoding said polypeptide/oligomer administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits.

When administered, the therapeutic preparations of the invention are applied in therapeutically-acceptable amounts and in pharmaceutically-acceptable compositions. The term “pharmaceutically acceptable” means a non-toxic material that does not interfere with the effectiveness of the biological activity of the active ingredients. Such preparations may routinely contain salts, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention.

Polypeptide/oligomer polypeptide compositions may be combined, if desired, with a pharmaceutically-acceptable carrier. The term “pharmaceutically-acceptable carrier” as used herein means one or more compatible solid or liquid fillers, diluents or encapsulating substances which are suitable for administration into a human. The term “carrier” denotes an organic or inorganic ingredient, natural or synthetic, with which the active ingredient is combined to facilitate the application.

The pharmaceutical compositions may contain suitable buffering agents, including: acetic acid in a salt; citric acid in a salt; boric acid in a salt; and phosphoric acid in a salt.

The pharmaceutical compositions also may contain, optionally, suitable preservatives, such as: benzalkonium chloride; chlorobutanol; parabens and thimerosal.

The pharmaceutical compositions may conveniently be presented in unit dosage form and may be prepared by any of the methods well-known in the art of pharmacy. All methods include the step of bringing the active agent into association with a carrier which constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for oral administration may be presented as discrete units, such as capsules, tablets, lozenges, each containing a predetermined amount of the active compound. Other compositions include suspensions in aqueous liquids or non-aqueous liquids such as a syrup, elixir or an emulsion.

Compositions suitable for parenteral administration conveniently comprise a sterile aqueous or non-aqueous preparation of polypeptides/oligomer or nucleic acids, which is preferably isotonic with the blood of the recipient. This preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono-or di-glycerides. In addition, fatty acids such as oleic acid may be used in the preparation of injectables.

Carrier formulation suitable for oral, subcutaneous, intravenous, intramuscular, etc. administrations can be found in Remington's Pharmaceutical Sciences, Mack Publishing Co., Easton, Pa.

In a preferred embodiment of the invention said pharmaceutical compositions modulates angiogenesis. Preferably said modulation is the inhibition of angiogenesis.

Preferably said inhibition relates to endothelial cell stimulated angiogenesis.

Alternatively, or preferably, said inhibition is the inhibition of macrophage and/or tumour cell stimulated angiogenesis.

In a further preferred embodiment of the invention said inhibition is mediated by the inhibition of pro-angiogenic factors. Ideally these are either intracellular or cell surface receptors.

More preferably still, said inhibition is mediated via inhibition of the activity of pro-angiogenic growth factors. Ideally said growth factors are selected from: VEGF, bFGF; aFGF; TGFβ; PDGF.

According to a yet further aspect of the invention there is provided the use of a polypeptide according to the invention, or part thereof, and/or oligomers for the manufacture of a medicament for use in the treatment of cancer.

According to a further aspect of the invention there is provided a nucleic acid molecule comprising DNA sequences selected from:
i) the DNA sequence as represented in FIG. 2;
ii) the DNA sequence as represented in FIG. 2 which has been modified by addition, deletion, or substitution of at least one nucleotide base within at least one codon to encode a modified peptide according to the invention;
iii) DNA sequences which hybridise to the sequences presented in FIG. 2 which encode a peptide having anti-angiogenic activity; and
iv) DNA sequences which are degenerate as a result of the genetic code to the DNA sequences defined in (i), (ii) or (iii).

In a preferred embodiment of the invention there is provided an isolated nucleic acid molecule which anneals under stringent hybridisation conditions to the sequences described in (i), (ii), (iii) and (iv) above.

Stringent hybridisation/washing conditions are well known in the art. For example, nucleic acid hybrids that are stable after washing in 0.1×SSC, 0.1% SDS at 60° C. It is well known in the art that optimal hybridisation conditions can be calculated if the sequence of the nucleic acid is known. Typically, hybridisation conditions uses 4-6×SSPE (20×SSPE contains 175.3 g NaCl, 88.2 g NaH₂PO₄H₂O and 7.4 g EDTA dissolved to 1 litre and the pH adjusted to 7.4); 5-10× Denhardts solution (50× Denhardts solution contains 5 g Ficoll (Type 400, Pharmacia), 5 g polyvinylpyrrolidone abd 5 g bovine serum albumen; 1001 g-1.0 mg/ml sonicated salmon/herring DNA; 0.1-1.0% sodium dodecyl sulphate; optionally 40-60% deionised formamide. Hybridisation temperature will vary depending on the GC content of the nucleic acid target sequence but will typically be between 420-65° C.

The present invention further provides, in another preferred embodiment, an isolated nucleic acid molecule consisting of a DNA sequence encoding one or more of the peptide amino acid sequences as disclosed herein.

Polypeptides according to the invention can be manufactured by in vitro peptide synthesis using standard peptide synthesis techniques. Alternatively, or preferably, polypeptides can be manufactured by recombinant techniques which are well known in the art.

According to a further aspect of the invention there is provided a vector, wherein said vector includes a nucleic acid molecule which encodes for polypeptides and/or oligomers according to the invention. In a preferred embodiment, the vector encodes one or more of the peptide amino acid sequences as disclosed herein.

Alternatively, vector(s) which include nucleic acid encoding said polypeptides can be adapted for recombinant expression.

In a preferred embodiment of the invention said vector is an expression vector adapted for prokaryotic or eukaryotic cell expression. Preferably said eukaryotic vector is adapted for gene therapy.

Typically said adaptation includes, by example and not by way of limitation, the provision of transcription control sequences (promoter sequences) which mediate cell/tissue specific expression. These promoter sequences may be cell/tissue specific, inducible or constitutive.

Promoter is an art recognised term and, for the sake of clarity, includes the following features which are provided by example only, and not by way of limitation. Enhancer elements are cis acting nucleic acid sequences often found 5′ to the transcription initiation site of a gene (enhancers can also be found 3′ to a gene sequence or even located in intronic sequences and is therefore position independent). Enhancers function to increase the rate of transcription of the gene to which the enhancer is linked. Enhancer activity is responsive to trans acting transcription factors (polypeptides) which have been shown to bind specifically to enhancer elements. The binding/activity of transcription factors (please see Eukaryotic Transcription Factors, by David S Latchman, Academic Press Ltd, San Diego) is responsive to a number of environmental cues which include, by example and not by way of limitation, intermediary metabolites or environmental effectors.

Promoter elements also include so called TATA box and RNA polymerase initiation selection (RIS) sequences which function to select a site of transcription initiation.

These sequences also bind polypeptides which function, inter alia, to facilitate transcription initiation selection by RNA polymerase.

Adaptations also include the provision of selectable markers and autonomous replication sequences which both facilitate the maintenance of said vector in either the eukaryotic cell or prokaryotic host. Vectors which are maintained autonomously are referred to as episomal vectors.

Adaptations which facilitate the expression of vector encoded genes include the provision of transcription termination/polyadenylation sequences. This also includes the provision of internal ribosome entry sites (IRES) which function to maximise expression of vector encoded genes arranged in bicistronic or multi-cistronic expression cassettes.

These adaptations are well known in the art. There is a significant amount of published literature with respect to expression vector construction and recombinant DNA techniques in general. Please see, Sambrook et al (1989) Molecular Cloning: A Laboratory Manual, Cold Spring Harbour Laboratory, Cold Spring Harbour, N.Y. and references therein; Marston, F (1987) DNA Cloning Techniques: A Practical Approach Vol m IRL Press, Oxford UK; DNA Cloning: F M Ausubel et al, Current Protocols in Molecular Biology, John Wiley & Sons, Inc. (1994).

In a yet further preferred embodiment of the invention there is provided a gene therapy vector comprising the nucleic acid according to the invention.

It will be apparent to one skilled in the art that the delivery of gene therapy vectors either to endothelial cells or tumour cells target the production of polypeptides according to the invention to the vicinity of the tumour thereby augmenting the anti-angiogenic effect of said polypeptides.

According to a yet further aspect of the invention there is provided a cell transformed/transfected with the nucleic acid according to the invention. Ideally said nucleic acid is the vector according to the invention.

According to a further aspect of the invention there is provided a method for the production of polypeptides according to the invention comprising:
i) providing a cell according to the invention;
ii) providing conditions conducive to the manufacture of polypeptides according to the invention; and
iii) purifying said polypeptides from a cell, or a cells culture environment.

According to yet still a further aspect of the invention there is provided a non-human, transgenic animal characterised in that said animal incorporates a nucleic acid molecule encoding a polypeptide according to the invention into its genome.

It will be apparent to one skilled in the art that the provision of non-human transgenic animals genetically modified by the provision of a transgene(s) encoding polypeptides according to the invention is an alternative source of active polypeptide. It is well known in the art that transgenic animals can be used to make various therapeutic polypeptides.

In a preferred embodiment of the invention said transgene is of human origin.

In a further aspect of the invention there is provided a method to treat an animal which would benefit from inhibition of angiogenesis comprising:
i) administering an effective amount of an agent comprising a polypeptide according to the invention, to an animal to be treated;
ii). monitoring the effects of said agent on the inhibition of angiogenesis.

In a preferred method of the invention said treatment is the inhibition of tumour development.

In an alternative method of treatment, polypeptides according to the invention are additionally conjugated, associated or crosslinked to an agent which augments the anti-angiogenic effect of the polypeptide/oligomer.

Typically the agent could be a cytotoxic agent, another anti-angiogenic agent, a prodrug activating enzyme, a chemotherapeutic agent, a pro-coagulant agent or immunomodulatory factor.

Examples of these are well known in the art, for example, and not by way of limitation cytotoxins, such as ricin A-chain or diphtheria toxin; antagonists of the key pro-angiogenic factors in tumours (eg VEGF, bFGF, TNF alpha, PDGF) would include neutralising antibodies or receptors for these factors, or tyrosine kinase inhibitors for their receptors (eg. PTK787 for the VEGF receptor, Flk-1/KDR); prodrug activating enzymes such as, human simplex virus-thymidine kinase HSV-TK, which activates the prodrug, ganciclovir when it is then admininistered systemically; chemotherapeutic agents, such as neocarzinostatin; cisplatin; carboplatin; cyclosphosphamide; melphalan; carmusline; methotrexate; 5-fluorouracil; cytarabine; mercaptopurine; daunorubicin; doxorubicin; epirubicin; vinblastine; vincristine; dactinomycin; mitomycin C; taxol; L-asparaginase; G-CSF; an enediyne such as chalicheamicin or esperamicin; chlorambucil; ARA-C; vindesine; bleomycin; and etoposide.

In addition, or alternatively, the cell surface domain of human tissue factor (this truncated form of tissue factor (tTF) could also be associated with polypeptides according to the invention. Truncated TF has limited anti-endothelial activity when free in the circulation, but becomes an effective and selective thrombogen (eg it causes extensive thrombosis and coagulation in blood vessels) when targeted to the surface of tumor endothelial cells.

An example of an immunomodulatory factor is the Fc effector domain of human IgG1. This binds natural killer (NK) cells and also the C1q protein that initiates the complement cascade. NK cells and complement then activate a powerful cytolytic response against the targeted endothelial cells.

It will be apparent that the above combinations of polypeptides and therapeutic agents will also have benefit with respect to the treatment of other conditions/diseases which are dependent on angiogenesis. For example, neovascular glaucoma, rheumatoid arthritis, psoriasis and diabetic retinopathy.

In a yet further alternative method of treatment, said gene therapy vector includes, and therefore said nucleic acid encoding a polypeptide according to the invention is provided with, nucleic acid encoding an agent which augments the anti-angiogenic effect of said polypeptide.

According to a yet further aspect of the invention there is provided an imaging agent comprising a polypeptide according to the invention. Examples of suitable imaging agents include, but are not limited to, contrast agents, magnetic resonance imaging agents, a paramagnetic isotope, a heavy metal, or a radioisotope.

It will be apparent to the skilled artisan that polypeptides according to the invention can be used to target imaging agents to, for example, tumours, to identify developing tumours or to monitor the effects of treatments to inhibit tumour growth. It will also be apparent that the combined therapeutic compositions which comprise both polypeptides and a further anti-angiogenic agent may be further associated with an imaging agent to monitor the distribution of the combined therapeutic composition and/or to monitor the efficacy of said combined composition.

Methods used to detect imaging agents are well known in the art and include, by example and not by way of limitation, positron emission tomographic detection of F¹⁸ and C¹¹ compounds.

According to a further aspect of the invention there is provided a screening method for the identification of ligands which modulate the interaction of a polypeptide/oligomer according to the invention with a vitronectin receptor.

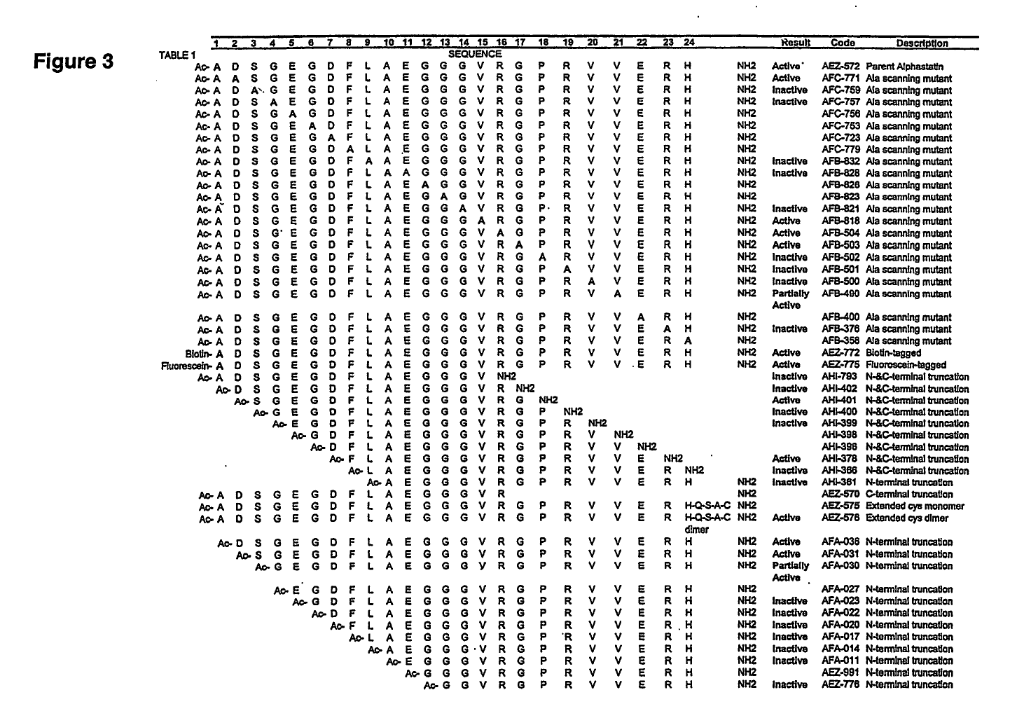

In a preferred method of the invention said method comprises the steps of:
i) providing a polypeptide comprising the amino acid sequence presented in FIG. 3 or active binding fragment thereof;
ii) providing at least one polypeptide or agent comprising an amino acid sequence selected from the sequences disclosed herein;
iii) providing at least one ligand to be tested;
iv) forming a preparation of (i), (ii) and (iii); and
v) detecting or measuring the effect of the ligand in (iii) on the interaction of the polypeptide in (i) with the polypeptide or agent in (ii).

In a further preferred method of the invention said ligand is pre-incubated with polypeptide in (i) prior to addition of the polypeptide or agent in (ii).

In an alternative preferred method of the invention said ligand is pre-incubated with the polypeptide or agent in (ii) prior to addition to the polypeptide in (i).

In a further preferred method of the invention said vitronectin receptor is presented in a soluble form or by a cell. Preferably said cell naturally expresses the vitronectin receptor. Examples of cells which naturally express the vitronectin receptor are endothelial cells, smooth muscle cells, osteoclasts and tumour cells. Alternatively said cell does not naturally express the vitronectin receptor, in which case said cells are preferably genetically engineered to express the vitronectin receptor.

According to a further aspect of the invention there are provided agent(s) identified by the screening method according to the invention. Preferably said agent(s) interfere with the interaction of polypeptides/oligomers with vitronectin receptor binding activity. Alternatively said agent promotes the interaction of polypeptides with vitronectin receptor binding activity.

An embodiment of the invention will now be described, by example only, and with reference to the following figures:

FIG. 1 represents the amino acid sequence of α1-24;

FIG. 2 represents the nucleic acid sequence encoding α1-24;

FIG. 3 represents a summary of the anti-angiogenic activity of polypeptides as herein disclosed.

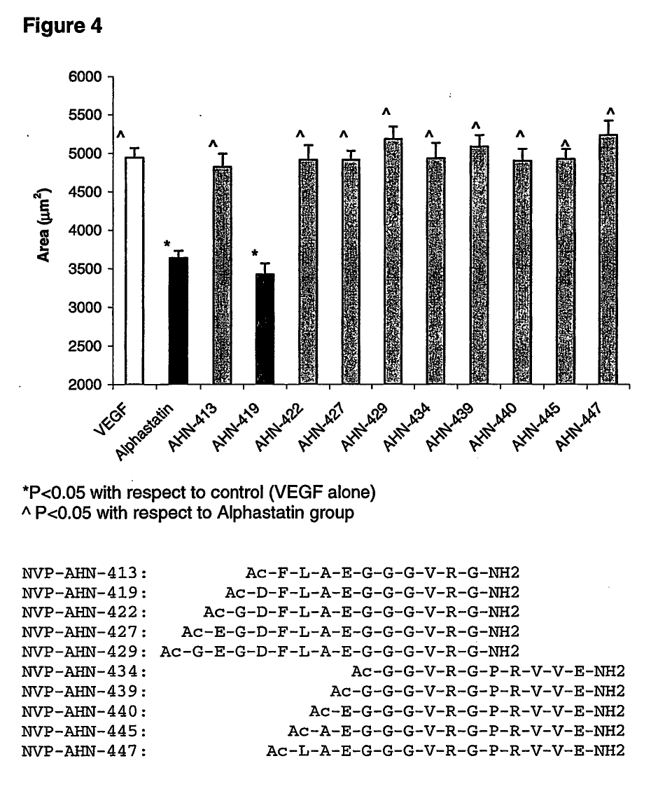

FIGS. 4 and 5 show tubule formation activity of peptides according to this invention.

Materials and Methods

Adult human dermal microvascular endothelial cells (HuDMECs) were obtained commercially (TCS Biologicals, Buckinghamshire, United Kingdom) and cultured in microvascular endothelial cell growth medium (EGM). This medium contains heparin (10 ng/ml), hydrocortisone, human epidermal growth factor (10 ng/ml), human fibroblast growth factor (10 ng/ml) (such endothelial growth factors are necessary for routine passaging of HuDMECs in culture) and dibutyryl cyclic AMP. This was supplemented with 5% heat-inactivated FCS, 50% g/ml gentamicin and 50 ng/ml amphotericin B (TCS Biologicals, United Kingdom). Murine endothelial cells (SVEC 4-10) were obtained from the ATCC and cultured in DMEM+10% FCS. Cells were grown at 37° C. in a 100% humidified incubator with a gas phase of 5% CO₂ and routinely screened for Mycoplasma. Prior to their use in the assays indicated below, HuDMECs were grown to 80% confluency, incubated in DMEM+1% FCS for 2h, then harvested with 0.05% trypsin solution, washed twice and resuspended to the cell density required for each assay (see below).

Peptide Synthesis and Analysis

Experimental Conditions for Analytical HPLC

Gradient 1: linear gradient over 2.5 min of MeCN/0.09% TFA and H₂O/0.1% TFA from 1:49 to 3:2; flow rate 4.0 mL/min, detection at 215 nm; SB-C18 ZORBAX, column 3.5 μm, 50×4.6 mm.

Gradient 2: linear gradient over 2.5 min of MeCN/0.09% TFA and H₂O/0.1% TFA from 1:49 to 1:0; flow rate 4.0 mL/min, detection at 215 nm; SB-C18 ZORBAX column, 3.5 μm, 50×4.6 mm.

Gradient 3: linear gradient over 2.5 min of MeCN/0.09% TFA and H₂O/0.1% TFA from 1:49 to 3:2; flow rate 4.0 mL/min, detection at 215 nm; Nucleosil C8 column, 5.0 μm, 125×4.0 mm.

Gradient 4: linear gradient from over 2.5 min of MeCN/0.09% TFA and H₂O/0.1% TFA from 1:49 to 3:2; flow rate 4 ml/min; detection at 215 nm; Chromolith SpeedROD C₁₈ column, 50×4.6 mm.

Gradient 5: linear gradient from over 2.5 min of MeCN/0.09% TFA and H₂O/0.1% TFA from 1:49 to 1:0; flow rate 4 ml/min; detection at 215 nm; Chromolith SpeedROD C₁₈ column, 50×4.6 mm.

Gradient 6: linear gradient from over 1.75 min of MeCN/0.09% TFA and H₂O/0.1% TFA from 1:49 to 1:0 and 0.75 min at 1:0; flow rate 4 ml/min; detection at 215 nm; Chromolith SpeedROD C₁₈ column, 50×4.6 mm.

Gradient 7: linear gradient from over 2.5 min of MeCN/0.09% TFA and H₂O/0.1% TFA from 1:49 to 1:0; flow rate 4 ml/min; detection at 215 nm; UP50 DB-5M Uptisphere C₁₈ column, 5.0 μm, 50×4.6 mm.

Gradient 8: linear gradient over 7 min of MeCN/0.09% TFA and H₂O/0.1% TFA from 1:49 to 1:0 and 3 min at 1:0; flow rate 2.0 mL/min, detection at 215 nm; SMT C₁₈ column, 5 μm, 250×4.6 mm.

Gradient 9: linear gradient over 10 min of MeCN/0.09% TFA and H₂O/0.1% TFA from 1:49 to 1:0; flow rate 2.0 mL/min, detection at 215 nm; SMT C₁₈ column, 5 μm, 250×4.6 mm.

t_{R}: retention time

NVP-AFC771-AI-17/CGE-3133

### EXAMPLE 1

### Ac-Ala-Ala-Ser-Gly-Glu-Gly-Asp-Phe-Leu-Ala-Glu-Gly-Gly-Gly-Val-Arg-Gly-Pro-Arg-Val-Val-Glu-Arg-His-NH₂ TFA Salt

The title peptide is synthesised on a Milligen 9050 automated peptide synthesizer (continuous flow; Millipore, Bedford, Mass., USA), starting with an Fmoc-PAL-PEG-PS resin (see Albericio, F. et al, J. Org. Chem., 55 (1990) 3730-3743) for establishing the C-terminal carboxamide, and using chemical protocols based on the fluorenylmethoxycarbonyl chemistry (see E. Atherton and R. C. Sheppard, in Solid-Phase Peptide Synthesis-A Practical Approach, eds: R. Rickwood and B. D. Hames, IRL Press at Oxford University Press, Oxford, 19989). The required Fmoc-amino acids (3 equiv.) are incorporated using their 2,4,5-trichlorophenyl esters (single coupling) with minimum reaction times of 30 min (see 9050 Plus PepSynthesizer User's Guide, Millipore Corporation, Bedford, Mass., 1992). Side chains are protected with the following groups: tert-butyl for aspartic acid, glutamic acid and serine; 2,2,5,7,8-pentamethyl-chroman-6-sulfonyl for arginine; and trityl for histidine. If required, a double coupling is performed using the Fmoc-amino acid (3 equiv.) and O-(1,2-dihydro-2-oxo-1-pyridyl)-1,1,3,3-tetramethyluronium tetrafluoroborate (3 equiv.) in the presence of diisopropylethylamine (6 equiv.). The complete peptide resin obtained after the final coupling reaction is simultaneously deprotected and cleaved by treatment with trifluoroacetic acid/water (95:5, v/v) for 3 h at room temperature. The filtrate from the cleavage reaction is precipitated in diisopropyl ether-petroleum ether (1:1, v/v) at 0° C., and the precipitate is collected by filtration. The crude peptide is purified by reversed-phase medium-pressure liquid chromatography using a C₁₈ column eluted with an acetonitrile-water gradient containing 0.1% trifluoroacetic acid (Merck LICHROPREP RP-18, 15-25 μm bead diameter, reversed phase column material based on C₁₈-derivatised silicagel, Merck, Darmstadt, FRG; column length 46 cm, diameter 3.6 cm; flow rate 53.3 ml/min; detection at 215 nm). Mass spectrometric analysis (matrix-assisted laser-desorption ionization time-of-flight mass spectrometry, MA/DI-TOF) of the purified compound reveals molecular masses within 0.1% of the expected value (negative ion mode): 2463.3 (calc. 2463.7, C₁₀₄H₁₆₅N₃₆O₃₄). The purity of the title compound is verified by reversed-phase analytical HPLC: single peak at t_{R}=1.81 min (Gradient 1); t_{R}=1.41 min (Gradient 2); and t_{R}=2.08 min (Gradient 3).

The following peptides are synthesised as described in Example 1.

NVP-AFC759-AI-1/CGE-3132

### EXAMPLE 2

### Ac-Ala-Asp-Ala-Gly-Glu-Gly-Asp-Phe-Leu-Ala-Glu-Gly-Gly-Gly-Val-Arg-Gly-Pro-Arg-Val-Val-Glu-Arg-His-NH₂ TFA Salt

Title compound: Mass spectral analysis (negative-ion mode): 2491.8 (calc. 2491.7, C₁₀₅H₁₆₅N₃₆O₃₅),

Analytical HPLC: t_{R}=1.83 min (Gradient 1); t_{R}=1.41 min (Gradient 2); and t_{R}=2.08 min (Gradient 3).

NVP-AFC757-AI-1/CGE-3131

### EXAMPLE 3

### Ac-Ala-Asp-Ser-Ala-Glu-Gly-Asp-Phe-Leu-Ala-Glu-Gly-Gly-Gly-Val-Arg-Gly-Pro-Arg-Val-Val-Glu-Arg-His-NH₂ TFA Salt

Title compound: Mass spectral analysis (negative-ion mode): 2521.8 (calc. 2521.7, C₁₀₆H₁₆₇N₃₆O₃₆),

Analytical HPLC: t_{R}=1.84 min (Gradient 1); t_{R}=1.42 min (Gradient 2); and t_{R}=2.10 min (Gradient 3).

NVP-AFC756-AI-1/CGE-3130

### EXAMPLE 4

### Ac-Ala-Asp-Ser-Gly-Ala-Gly-Asp-Phe-Leu-Ala-Glu-Gly-Gly-Gly-Val-Arg-Gly-Pro-Arg-Val-Val-Glu-Arg-His-NH₂ TFA

Title compound: Mass spectral analysis (negative-ion mode): 2449.6 (calc. 2449.7, C₁₀₃H₁₆₃N₃₆O₃₄),

Analytical HPLC: t_{R}=1.84 min (Gradient 1); t_{R}=1.41 min (Gradient 2); and t_{R}=2.09 min (Gradient 3).

NVP-AFC753-AI-1/CGE-3129

### EXAMPLE 5

### Ac-Ala-Asp-Ser-Gly-Glu-Ala-Asp-Phe-Leu-Ala-Glu-Gly-Gly-Gly-Val-Arg-Gly-Pro-Arg-Val-Val-Glu-Arg-His-NH₂ TFA Salt

Title compound: Mass spectral analysis (negative-ion mode): 2522.0 (calc. 2521.7, C₁₀₆H₁₆₇N₃₆O₃₆),

Analytical HPLC: t_{R}=1.85 min (Gradient 1); t_{R}=1.43 min (Gradient 2); and t_{R}=2.11 min (Gradient 3).

NVP-AFC723-AI-1/CGE-3128

### EXAMPLE 6

### Ac-Ala-Asp-Ser-Gly-Glu-Gly-Ala-Phe-Leu-Ala-Glu-Gly-Gly-Gly-Val-Arg-Gly-Pro-Arg-Val-Val-Glu-Arg-Iis-NH₂ TFA Salt

Title compound: Mass spectral analysis (negative-ion mode): 2463.1 (calc. 2463.7, C₁₀₄H₁₆₅N₃₆O₃₄).

Analytical HPLC: t_{R}=1.82 min (Gradient 1); t_{R}=1.41 min (Gradient 2); and t_{R}=2.07 min (Gradient 3).

NVP-AFC779-AI-1/CGE-3127

### EXAMPLE 7

### Ac-Ala-AspSer-Gly-Glu-Gly-Asp-Ala-Leu-Ala-Glu-Gly-Gly-Gly-Val-Arg-Gly-Pro-Arg-Val-Val-Glu-Arg-His-NH₂ TFA Salt

Title compound: Mass spectral analysis (negative-ion mode): 2431.7 (calc. 2431.6, C₉₉H₁₆₁N₃₆O₃₆),

Analytical HPLC: t_{R}=1.66 min (Gradient 1); and t_{R}=1.94 min (Gradient 3).

NVP-AFB832-AI-1/CGE-3121

### EXAMPLE 8

### Ac-Ala-Asp-Ser-Gly-Glu-Gly-Asp-Phe-Ala-Ala-Glu-Gly-Gly-Gly-Val-Arg-Gly-Pro-Arg-Val-Val-Glu-Arg-His-NH₂ TFA Salt

Title compound: Mass spectral analysis (negative-ion mode): 2465.6 (calc. 2465.6, C₁₀₂H₁₅₉N₃₆O₃₆),

Analytical HPLC: t_{R}=1.66 min (Gradient 1); and t_{R}=1.94 min (Gradient 3).

NVP-AFB828-AI-1/CGE-3120

### EXAMPLE 9

### Ac-Ala-Asp-Ser-Gly-Glu-Gly-Asp-Phe-Leu-Ala-Ala-Gly-Gly-Gly-Val-Arg-Gly-Pro-Arg-Val-Val-Glu-Arg-His-NH₂ TFA Salt

Title compound: Mass spectral analysis (negative-ion mode): 2449.1 (calc. 2449.7, C₁₀₃H₁₆₃N₃₆O₃₄).

Analytical HPLC: t_{R}=1.91 min (Gradient 1); t_{R}=1.83 min (Gradient 4); and t_{R}=1.44 min (Gradient 5).

NVP-AFB826-AI-1/CGE-3119

### EXAMPLE 10

### Ac-Ala-Asp-Ser-Gly-Glu-Gly-Asp-Phe-Leu-Ala-Glu-Ala-Gly-Gly-Val-Arg-Gly-Pro-Arg-Val-Val-Glu-Arg-His-NH₂ TFA Salt

Title compound: Mass spectral analysis (negative-ion mode): 2521.7 (calc. 2521.7, C₁₀₆H₁₆₇N₃₆O₃₆).

Analytical HPLC: t_{R}=1.85 min (Gradient 1); t_{R}=1.88 min (Gradient 4); and t_{R}=1.47 min (Gradient 5).

NVP-AFB823-AI-1/CGE-3115

### EXAMPLE 11

### Ac-Ala-Asp-Ser-Gly-Glu-Gly-Asp-Phe-Leu-Ala-Glu-Gly-Ala-Gly-Val-Arg-Gly-Pro-Arg-Val-Val-Glu-Arg-His-NH2 TFA Salt

Title compound: Mass spectral analysis (negative-ion mode): 2521.7 (calc. 2521.7, C₁₀₆H₁₆₇N₃₆O₃₆).

Analytical HPLC: t_{R}=1.82 min (Gradient 1); t_{R}=1.84 min (Gradient 4); and t_{R}=1.44 min (Gradient 5).

NVP-AFB821-AI-1/CGE-3114

### EXAMPLE 12

### Ac-Ala-Asp-Ser-Gly-Glu-Gly-Asp-Phe-Leu-Ala-Glu-Gly-Gly-Ala-Val-Arg-Gly-Pro-Arg-Val-Val-Glu-Arg-His-NH₂ TFA Salt

Title compound: Mass spectral analysis (negative-ion mode): 2521.7 (calc. 2521.7, C₁₀₆H₁₆₇N₃₆O₃₆).

Analytical HPLC: t_{R}=1.80 min (Gradient 1); t_{R}=1.83 min (Gradient 4); and t_{R}=1.43 min (Gradient 5).

NVP-AFB818-AI-1/CGE-3113

### EXAMPLE 13

### Ac-Ala-Asp-Ser-Gly-Glu-Gly-Asp-Phe-Leu-Ala-Glu-Gly-Gly-Gly-Ala-Arg-Gly-Pro-Arg-Val-Val-Glu-Arg-His-NH₂ TFA Salt

Title compound: Mass spectral analysis (negative-ion mode): 2479.7 (calc. 2479.7, C₁₀₃H₁₆₁N₃₆O₃₆).

Analytical HPLC: t_{R}=1.76 min (Gradient 1); t_{R}=1.78 min (Gradient 4); and t_{R}=1.41 min (Gradient 5).

NVP-AFB504-AI-1/CGE-3110

### EXAMPLE 14

### Ac-Ala-Asp-Ser-Gly-Glu-Gly-Asp-Phe-Leu-Ala-Glu-Gly-Gly-Gly-Val-Ala-Gly-Pro-Arg-Val-Val-Glu-Arg-His-NH₂ TFA Salt

Title compound: Mass spectral analysis (negative-ion mode): 2422.6 (calc. 2422.6, C₁₀₂H₁₅₈N₃₃O₃₆).

Analytical HPLC: t_{R}=1.79 min (Gradient 4); and t_{R}=1.43 min (Gradient 5).

NVP-AFB503-AI-1/CGE-3109

### EXAMPLE 15

### Ac-Ala-Asp-Ser-Gly-Glu-Gly-Asp-Phe-Leu-Ala-Glu-Gly-Gly-Gly-Val-Arg-Ala-Pro-Arg-Val-Val-Glu-Arg-His-NH₂ TFA Salt

Title compound: Mass spectral analysis (negative-ion mode): 2521.1 (calc. 2521.7, C₁₀₆H₁₆₇N₃₆O₃₆).

Analytical HPLC: t_{R}=1.77 min (Gradient 4); and t_{R}=1.41 min (Gradient 5).

NVP-AFB502-AI-1/CGE-3108

### EXAMPLE 16

### Ac-Ala-Asp-Ser-Gly-Glu-Gly-Asp-Phe-Leu-Ala-Glu-Gly-Gly-Gly-Val-Arg-Gly-Ala-Arg-Val-Val-Glu-Arg-His-NH₂ TFA Salt

Title compound: Mass spectral analysis (negative-ion mode): 2480.1 (calc. 2481.7, C₁₀₃H₁₆₃N₃₆O₃₆).

Analytical HPLC: t_{R}=1.74 min (Gradient 4); and t_{R}=1.40 min (Gradient 5).

NVP-AFB501-AI-1/CGE-3105

### EXAMPLE 17

### Ac-Ala-Asp-Ser-Gly-Glu-Gly-Asp-Phe-Leu-Ala-Glu-Gly-Gly-Gly-Val-Arg-Gly-Pro-Ala-Val-Val-Glu-Arg-His-NH₂ TFA Salt

Title compound: Mass spectral analysis (negative-ion mode): 2422.3 (calc. 2422.6, C₁₀₂H₁₅₈N₃₃O₃₆).

Analytical HPLC: t_{R}=1.76 min (Gradient 1); t_{R}=1.79 min (Gradient 4); and t_{R}=1.43 min (Gradient 5).

NVP-AFB500-AI-I/CGE-3104

### EXAMPLE 18

### Ac-Ala-Asp-Ser-Gly-Glu-Gly-Asp-Phe-Leu-Ala-Glu-Gly-Gly-Gly-Val-Arg-Gly-Pro-Arg-Ala-Val-Glu-ArgHis-NH₂ TFA Salt

Title compound: Mass spectral analysis (negative-ion mode): 2481.0 (calc. 2479.7, C₁₀₃H₁₆₁N₃₆O₃₆).

Analytical HPLC: t_{R}=1.72 min (Gradient 4); and t_{R}=1.38 min (Gradient 5).

NVP-AFB490-AI-1/CGE-3103

### EXAMPLE 19

### Ac-Ala-Asp-Ser-Gly-Glu-Gly-Asp-Phe-Leu-Ala-Glu-Gly-Gly-Gly-Val-Arg-Gly-Pro-Arg-Val-Ala-Glu-Arg-His-NH₂ TFA Salt

Title compound: Mass spectral analysis (negative-ion mode): 2481.7 (calc. 2479.7, C₁₀₃H₁₆₁N₃₆O₃₆).

Analytical HPLC: t_{R}=1.72 min (Gradient 4); and tR-1.38 min (Gradient 5).

NVP-AFB400-AI-1/CGE-3100

### EXAMPLE 20

### Ac-Ala-Asp-Ser-Gly-Glu-Gly-Asp-Phe-Leu-Ala-Glu-Gly-Gly-Gly-Val-Arg-Gly-Pro-Arg-Val-Val-Ala-Arg-His-NH₂ TFA Salt

Title compound: Mass spectral analysis (negative-ion mode): 2450.0 (calc. 2449.7, C₁₀₃H₁₆₃N₃₆O₃₄).

Analytical HPLC: t_{R}=1.77 min (Gradient 4); and t_{R}=1.40 min (Gradient 5).

NVP-AFB376-AI-1/CGE-3099

### EXAMPLE 21

### Ac-Ala-Asp-Ser-Gly-Glu-Gly-Asp-Phe-Leu-Ala-Glu-Gly-Gly-Gly-Val-Arg-Gly-Pro-Arg-Val-Val-Glu-Ala-His-NH₂ TFA Salt

Title compound: Mass spectral analysis (negative-ion mode): 2421.0 (calc. 2422.6, C₁₀₂H₁₅₈N₃₃O₃₆).

Analytical HPLC: t_{R}=1.78 min (Gradient 4); and t_{R}=1.43 min (Gradient 5).

NVP-AFB358-AI-1/CGE-3098

### EXAMPLE 22

### Ac-Ala-Asp-Ser-Gly-Glu-Gly-Asp-Phe-Leu-Ala-Glu-Gly-Gly-Gly-Val-Arg-Gly-Pro-Arg-Val-Val-Glu-Arg-Ala-NH₂ TFA Salt

Title compound: Mass spectral analysis (negative-ion mode): 2442.1 (calc. 2441.7, C₁₀₂H₁₆₃N₃₄O₃₆).

Analytical HPLC: t_{R}=1.79 min (Gradient 4); and t_{R}=1.44 min (Gradient 5).

NVP-AEZ772-AI-1/2/CGE-2943

### EXAMPLE 23

### Biotin-8-amino-3,6-dioxaoctanoyl-Ala-Asp-Ser-Gly-Glu-Gly-Asp-Phe-Leu-Ala-Glu-Gly-Gly-Gly-Val-Arg-Gly-Pro-Arg-Val-Val-Glu-Arg-His-NH₂ TFA Salt

Fmoc-8-amino-3,6-dioxaoctanoic acid (3 equiv.; Neosystem, Strasbourg, France) is incorporated using O-(1,2-dihydro-2-oxo-1-pyridyl)-1,1,3,3-tetramethyluronium tetrafluoroborate (3 equiv.; single coupling) in the presence of diisopropylethylamine (6 equiv.). (+)-Biotin (3 equiv.; Fluka, Buchs, Switzerland) is coupled using O-(1,2-dihydro-2-oxo-1-pyridyl)-1,1,3,3-tetramethyluronium tetrafluoroborate (3 equiv.; first coupling) and N-[(dimethylamino)-1H-1,2,3,-triazolo-[4,5b]pyridin-1-yl-methylene]-N-methylmethanaminium hexafluorophosphate N-oxide (3 equiv.; second coupling) in the presence of diisopropylethyl amine (6 equiv.) Title compound: Mass spectral analysis (negative-ion mode): 2837.1 (calc. 2837.1, C₁₁₉H₁₈₈N₃₉O₄₀S₁).

Analytical HPLC: t_{R}=1.74 min (Gradient 4); and t_{R}=1.42 min (Gradient 5).

NVP-AEZ775-AI-1/2/3/CGE-2944

### EXAMPLE 24

### Fluorescein-8-amino-3,6-dioxaoctanoyl-Ala-Asp-Ser-Gly-Glu-Gly-Asp-Phe-Leu-Ala-Glu-Gly-Gly-Gly-Val-Arg-Gly-Pro-Arg-Val-Val-Glu-Arg-His-NH₂ TFA Salt

Fmoc-8-amino-3,6-dioxaoctanoic acid (3 equiv.; Neosystem, Strasbourg, France) is incorporated using O-(1,2-dihydro-2-oxo-1-pyridyl)-1,1,3,3-tetramethyluronium tetrafluoroborate (3 equiv.; single coupling) in the presence of diisopropylethylamine (6 equiv.). Fluoresceinisothiocyanate isomer I (3 equiv.; Fluka, Buchs, Switzerland) is incorporated to the N-terminal amino group in the presence of diisopropylethylamine (6 equiv.). Coupling is achieved by dissolving the building block and the base in N-methyl-2-pyrrolidone, adding the mixture to the resin, and shaking at room temperature for 21 h.

Title compound: Mass spectral analysis (negative-ion mode): 3000.6 (calc. 3000.2, C₁₃₀H₁₈₅N₃₈O₄₃S₁).

Analytical HPLC: t_{R}=2.00 min (Gradient 4); and t_{R}=1.63 min (Gradient 5).

NVP-AEZ776-AI-1/CGE-2957

### EXAMPLE 25

### Ac-Gly-Gly-Val-Arg-Gly-Pro-Arg-Val-Val-Glu-Arg-His-NH₂ TFA Salt

Title compound: Mass spectral analysis (negative-ion mode): 1358.3 (calc. 1358.6, C₅₇H₉₇N₂₄O₁₅).

Analytical HPLC: t_{R}=1.46 min (Gradient 4); and tR-1.13 min (Gradient 6).

NVP-AEZ991-AI-11CGE-2959

### EXAMPLE 26

### Ac-Gly-Gly-Gly-Val-Arg-Gly-Pro-Arg-Val-Val-Glu-Arg-His-NH₂ TFA Salt

Title compound: Mass spectral analysis (negative-ion mode): 1415.8 (calc. 1415.6, C₅₉H₁₀₀N₂₅O₁₆).

Analytical HPLC: t_{R}=1.45 min (Gradient 4); and t_{R}=1.11 min (Gradient 6).

NVP-AFA011-AI-1/CGE-2960

### EXAMPLE 27

### Ac-Glu-Gly-Gly-Gly-Val-Arg-Gly-Pro-Arg-Val-Val-Glu-Arg-His-NH₂ TFA Salt

Title compound: Mass spectral analysis (positive-ion mode): 1546.8 (calc. 1546.7, C₆₋₄Hlo₉N₂₆O₁₉).

Analytical HPLC: t_{R}=1.45 min (Gradient 4); and t_{R}=1.11 min (Gradient 6).

NVP-AFA014-AI-1/CGE-2961

### EXAMPLE 28

### Ac-Ala-Glu-Gly-Gly-Gly-Val-Arg-Gly-Pro-Arg-Val-Val-Glu-Arg-His-NH2 TPA Salt

Title compound: Mass spectral analysis (positive-ion mode): 1617.5 (calc. 1617.8, C₆₇H₁₁₄N₂₇O₂₀).

Analytical HPLC: t_{R}=1.47 min (Gradient 4); and t_{R}=1.11 min (Gradient 6).

NVP-AFA017-AI-1/CGE-2962

### EXAMPLE 29

### Ac-Leu-Ala-Glu-Gly-Gly-Gly-Val-Arg-Gly-Pro-Arg-Val-Val-Glu-Arg-His-NH2 TFA Salt

Title compound: Mass spectral analysis (positive-ion mode): 1731.6 (calc. 1731.0, C₇₂H₁₂₅N₂₈O₂₁).

Analytical HPLC: t_{R}=1.65 min (Gradient 4); and t_{R}=1.21 min (Gradient 6).

NVP-AFA020-AI-1/CGE-2963

### EXAMPLE 30

### Ac-Phe-Leu-Ala-Glu-Gly-Gly-Gly-Val-Arg-Gly-Pro-Arg-Val-Val-Glu-Arg-His-NH2

Title compound: Mass spectral analysis (positive-ion mode): 1878.2 (calc. 1878.2, C₈₂H₁₃₄N₂₉O₂₂).

Analytical HPLC: t_{R}=1.82 min (Gradient 4); and t_{R}=1.32 min (Gradient 6).

NVP-AFA022-AI-1/CGE-2964

### EXAMPLE 31

### Ac-Asp-Phe-Leu-Ala-Glu-Gly-Gly-Gly-Val-Arg-Gly-Pro-Arg-Val-Val-Glu-Arg-His-NH₂ TFA Salt

Title compound: Mass spectral analysis (negative-ion mode): 1990.9 (calc. 1991.2, C₈₆H₁₃₇N₃O₂₅).

Analytical HPLC: t_{R}=1.86 min (Gradient 4); and t_{R}=1.31 min (Gradient 6).

NVP-AFA023-AI-1/CGE-2965

### EXAMPLE 32

### Ac-Gly-Asp-Phe-Leu-Ala-Glu-Gly-Gly-Gly-Val-Arg-Gly-Pro-Arg-Val-Val-Glu-Arg-His-NH₂ TFA Salt

Title compound: Mass spectral analysis (positive-ion mode): 2049.7 (calc. 2050.3, C₈₈H₁₄₂N₃₁O₂₆).

Analytical HPLC: t_{R}=1.83 min (Gradient 4); and t_{R}=1.30 min (Gradient 6).

NVP-AFA027-AI-1/CGE-2966

### EXAMPLE 33

### Ac-Glu-Gly-Asp-Phe-Leu-Ala-Glu-Gly-Gly-Gly-Val-Arg-Gly-Pro-Arg-Val-Val-Glu-Arg-His-NH₂ TFA Salt

Title compound: Mass spectral analysis (positive-ion mode): 2179.3 (calc. 2179.4, C₉₃H₁₄₉N₃₂O₂₉).

Analytical HPLC: t_{R}=1.81 min (Gradient 4); and t_{R}=1.29 min (Gradient 6).

NVP-AFA030-AI-1/CGE-2967

### EXAMPLE 34

### Ac-Gly-Glu-Gly-Asp-Phe-Leu-Ala-Glu-Gly-Gly-Gly-Val-Arg-Gly-Pro-Arg-Val-Val-Glu-Arg-His-NH₂ TFA Salt

Title compound: Mass spectral analysis (positive-ion mode): 2234.5 (calc. 2234.5, C₉₅H₁₅₀N₃₃O₃₀).

Analytical HPLC: t_{R}=1.79 min (Gradient 4); and t_{R}=1.27 min (Gradient 6).

NVP-AFA031-AI-I/CGE-2968

### EXAMPLE 35

### Ac-Ser-Gly-Glu-Gly-Asp-Phe-Leu-Ala-Glu-Gly-Gly-Gly-Val-Arg-Gly-Pro-Arg-Val-Val-Glu-Arg-His-NH₂ TFA Salt

Tide compound: Mass spectral analysis (negative-ion mode): 2321.0 (calc. 2321.5, C₉₈H₁₅₅N₃₄O₃₂). Analytical HPLC: t_{R}=1.79 min (Gradient 4); and t_{R}=1.26 min (Gradient 6).

NVP-AFA036-AI-1/CGE-2969

### EXAMPLE 36

### Ac-Asp-Ser-Gly-Glu-Gly-Asp-Phe-Leu-Ala-Glu-Gly-Gly-Gly-Val-Arg-Gly-Pro-Arg-Val-Val-Glu-Arg-His-NH₂ TFA Salt

Title compound: Mass spectral analysis (negative-ion mode): 2436.3 (calc. 2436.6, C₁₀₂H₁₆₀N₃₅O₃₅).

Analytical HPLC: t_{R}=1.78 min (Gradient 4); and t_{R}=1.27 min (Gradient 6).

NVP-AEZ570-AI-1/CGE-2809

### EXAMPLE 37

### Ac-Ala-Asp-Ser-Gly-Glu-Gly-Asp-Phe-Leu-Ala-Glu-Gly-Gly-Gly-Val-Arg-NH2 TFA Salt

Title compound: Mass spectral analysis (negative-ion mode): 1576.5 (calc. 1576.6, C₆₅H₉₉N₂₀O₂₆).

Analytical HPLC: t_{R}=1.52 min (Gradient 7); and t_{R}=5.08 min (Gradient 8).

NVP-AEZ572-AI-1/2/CGE-2810

### EXAMPLE 38

### Ac-Ala-Asp-Ser-Gly-Glu-Gly-Asp-Phe-Leu-Ala-Glu-Gly-Gly-Gly-Val-Arg-Gly-Pro-Arg-Val-Val-Glu-Arg-His-NH₂ TFA Salt

Title compound: Mass spectral analysis (negative-ion mode): 2507.7 (calc. 2507.7, C₁₀₅H₁₆₅N₃₆O₃₆).

Analytical HPLC: t_{R}=1.87 min (Gradient 4); and t_{R}=1.49 min (Gradient 5).

NVP-AEZ575-AI-1/CGE-2811

### EXAMPLE 39

### Ac-Ala-Asp-Ser-Gly-Glu-Gly-Asp-Phe-Leu-Ala-Glu-Gly-Gly-Gly-Val-Arg-Gly-Pro-Arg-Val-Val-Glu-Arg-His-Gln-Ser-Ala-Cys-NH₂ TFA Salt

Title compound: Mass spectral analysis (negative-ion mode): 2898.0 (calc. 2897.1, C₁₁₉H₁₈₈N₄₁O₄₂S₁).

Analytical HPLC: t_{R}=5.01 min (Gradient 8); and t_{R}=5.76 min (Gradient 9).

NVP-AEZ576-AI-1/CGE-2827

### EXAMPLE 40

### (Ac-Ala-Asp-Ser-Gly-Glu-Gly-Asp-Phe-Leu-Ala-Glu-Gly-Gly-Gly-Val-Arg-Gly-Pro-Arg-Val-Val-Glu-Arg-His-Gln-Ser-Ala-Cys-NH₂)SS(Ac-Ala-Asp-Ser-Gly-Glu-Gly-Asp-Phe-Leu-Ala-Glu-Gly-Gly-Gly-Val-Arg-Gly-Pro-Arg-Val-Val-Glu-Arg-His-Gln-Ser-Ala-Cys-NH₂) TEA Salt

The peptide (50 mg), which is described in Example 39, is dissolved in a 1% solution of sodium carbonate (500 μl) and stirred at room temperature overnight. After this time, the crude peptide purified as indicated in Example 1.

Title compound: Mass spectral analysis (negative-ion mode): 5795.1 (calc. 5793.3, C₂₃₈H₃₇₅N₈₂O₈₄S₂).

Analytical HPLC: t_{R}=1.98 min (Gradient 4); and t_{R}=1.56 min (Gradient 5).

NVP-AHI361-AI-1/CGE-3348

### EXAMPLE 41

### Ac-Ala-Glu-Gly-Gly-Gly-Val-Arg-Gly-Pro-Arg-Val-Val-Glu-Arg-His-NH2 TFA Salt

Title compound: Mass spectral analysis (negative-ion mode): 1615.8 (calc. 1615.8, C₆₇H₁₁₂N₂₇O₂₀).

Analytical HPLC: t_{R}=1.58 min (Gradient 4); and t_{R}=1.29 min (Gradient 5).

NVP-AHI366-AI-1/CGE-3349

### EXAMPLE 42

### Ac-Leu-Ala-Glu-Gly-Gly-Gly-Val-Arg-Gly-Pro-Arg-Val-Val-Glu-Arg-NH₂ TFA Salt

Title compound: Mass spectral analysis (negative-ion mode): 1591.4 (calc. 1591.8, C₆₇H₁ 16N₂₅O₂₀).

Analytical HPLC: t_{R}=1.76 min (Gradient 4); and t_{R}=1.37 min (Gradient 5).

NVP-AHI378-AI-I/CGE-3350

### EXAMPLE 43

### Ac-Phe-Leu-Ala-Glu-Gly-Gly-Gly-Val-Arg-Gly-Pro-Arg-Val-Val-Glu-NH2 TFA Salt

Title compound: Mass spectral analysis (negative-ion mode): 1582.6 (calc. 1582.8, C₇₀H₁₁₃N₂₂O₂₀).

Analytical HPLC: t_{R}=2.03 min (Gradient 4); and t_{R}=1.59 min (Gradient 5).

NVP-AHI396-AI-1/CGE-3351

### EXAMPLE 44

### Ac-Asp-Phe-Leu-Ala-Glu-Gly-Gly-Gly-Val-Arg-Gly-Pro-Arg-Val-Val-NH₂ TFA Salt

Title compound: Mass spectral analysis (negative-ion mode): 1568.0 (calc. 1568.8, C₆₉H₁₁₁N₂₂O₂₀).

Analytical HPLC: t_{R}=2.03 min (Gradient 4); and t_{R}=1.55 min (Gradient 5).

NVP-AHI398-AI-1/CGE-3352

### EXAMPLE 45

### Ac-Gly-Asp-Phe-Leu-Ala-Glu-Gly-Gly-Gly-Val-Arg-Gly-Pro-Arg-Val-NH2 TFA Salt

Title compound: Mass spectral analysis (negative-ion mode): 1526.5 (calc. 1526.7, C₆₆H₁₁O₅N₂₂O₂₀).

Analytical HPLC: t_{R}=1.96 min (Gradient 4); and t_{R}=1.50 min (Gradient 5).

NVP-AHI399-AI-1/CGE-3353

### EXAMPLE 46

### Ac-Glu-Gly-Asp-Phe-Leu-Ala-Glu-Gly-Gly-Gly-Val-Arg-Gly-Pro-Arg-NH2 TFA Salt

Title compound: Mass spectral analysis (negative-ion mode): 1556.3 (calc. 1556.7, C₆₇H₁₀₅N₂₁O₂₂).

Analytical HPLC: t_{R}=1.86 min (Gradient 4); and t_{R}=1.48 min (Gradient 5).

NVP-AHI400-AI-1/CGE-3354

### EXAMPLE 47

### Ac-Gly-Glu-Gly-Asp-Phe-Leu-Ala-Glu-Gly-Gly-Gly-Val-Arg-Gly-Pro-NH2 TFA Salt

Title compound: Mass spectral analysis (negative-ion mode): 1457.3 (calc. 1457.6, C₆₂H₉₄N₁₉O₂₂).

Analytical HPLC: t_{R}=1.89 min (Gradient 4); and t_{R}=1.50 min (Gradient 5).

NVP-AHI401-AI-1/CGE-3355

### EXAMPLE 48

### Ac-Ser-Gly-Glu-Gly-Asp-Phe-Leu-Ala-Glu-Gly-Gly-Gly-Val-Arg-Gly-NH₂ TFA Salt

Title compound: Mass spectral analysis (negative-ion mode): 1447.3 (calc. 1447.5, C₆₀H₉₂N₁₉O₂₃).

Analytical HPLC: t_{R}=1.83 min (Gradient 4); and t_{R}=1.46 min (Gradient 5).

NVP-AHI402-AI-1/CGE-3356

### EXAMPLE 49

### Ac-Asp-Ser-Gly-Glu-Gly-Asp-Phe-Leu-Ala-Glu-Gly-Gly-Gly-Val-Arg-NH2 TFA Salt

Title compound: Mass spectral analysis (negative-ion mode): 1505.1 (calc. 1505.6, C₆₂H₉₄N₁₉O₂₅).

Analytical HPLC: t_{R}=1.84 min (Gradient 4); and t_{R}=1.49 min (Gradient 5).

NVP-AHI793-AI-1/CGE-3357

### EXAMPLE 50

### Ac-Ala-Asp-Ser-Gly-Glu-Gly-Asp-Phe-Leu-Ala-Glu-Gly-Gly-Gly-Val-NH2 TFA Salt

Title compound: Mass spectral analysis (negative-ion mode): 1420.6 (calc. 1420.5, C₅₉H₈₇N₁₆O₂₅).

Analytical HPLC: t_{R}=1.86 min (Gradient 4); and t_{R}=1.48 min (Gradient 5).

NVP-ANH419-AI-1, CGE-3459

### EXAMPLE 51

### Ac-Asp-Phe-Leu-Ala-Glu-Gly-Gly-Gly-Val-Arg-Gly-NH₂ TFA Salt

Title compound: Mass spectral analysis (negative-ion mode): 1117.2 (calc. 1117.2, C₄₈H₇₄N₁₅O₁₆).

Analytical HPLC, t_{R}=1.94 min (Gradient 4); t_{R}=1.58 min (Gradient 5).

NVP-AML639-NX-1, CGE-3639

### EXAMPLE 52

### Ac-Asp-Phe-Leu-Ala-Glu-Gly-Gly-Gly-Val-NH₂

Title compound: Mass spectral analysis (negative-ion mode): 903.6 (calc. 904.0, C₄₀H₅₉N₁₀O₁₄).

Analytical HPLC, t_{R}=1.97 min (Gradient 4); t_{R}=1.56 min (Gradient 5).

NVP-AML640-NX-1, CGE-3640

### EXAMPLE 53

### Ac-Asp-Phe-Leu-Ala-Glu-Gly-Gly-Gly-NH₂

Title compound: Mass spectral analysis (negative-ion mode): 804.5 (calc. 804.8, C₃₅H₅₀N₉O₁₃).

Analytical HPLC, t_{R}=1.87 min (Gradient 4); t_{R}=1.50 min (Gradient 5).

NVP-AML648-AI-1, CGE-3650

### EXAMPLE 54

### Ac-Asp-Phe-Leu-Ala-Glu-NHCH₂CH₂OCH₂CH₂OCH₂CO-Val-Arg-Gly-NH2 TFA Salt

Fmoc-8-amino-3,6-dioxaoctanoic acid is purchased from Neosystem (Strasbourg, France). Title compound: Mass spectral analysis (negative-ion mode): 1090.7 (calc. 1091.2, C₄₈H₇₆N₁₃O₁₆).

Analytical HPLC, t_{R}=2.03 min (Gradient 4); t_{R}=1.59 min (Gradient 5).

NVP-AML655-A-1, CGE-3651

### EXAMPLE 55

### Ac-Asp-Phe-Leu-Ala-Glu-Ala-Val-Arg-Gly-NH₂ TFA Salt

Fmoc-β-alanine is purchased from Fluka (Buchs, Switzerland). Title compound:

Mass spectral analysis (negative-ion mode): 1017.4 (calc. 1017.1, C₄₅H₇₀N₁₃O₁₄).

Analytical HPLC, t_{R}=1.99 min (Gradient 4); t_{R}=1.57 min (Gradient 5).

NVP-AML658-AI-1, CGE-3652

### EXAMPLE 56

### Ac-Asp-Phe-Leu-Ala-Glu-Ala-Ala-Val-Arg-Gly-NH₂ TFA Salt

Fmoc-β-alanine is purchased from Fluka (Buchs, Switzerland). Title compound:

Mass spectral analysis (negative-ion mode): 1088.4 (calc. 1088.2, C₄₈H₇₅N₁₄O₁₅).

Analytical HPLC, t_{R}=1.95 min (Gradient 4); t_{R}=1.54 min (Gradient 5).

NVP-AML632-AI-1 CGE-3638

### EXAMPLE 57

### Ac-Asp-Phe-Leu-Ala-Glu-Gly-Gly-Gly-Val-Arg-NH₂ TFA Salt

Title compound: Mass spectral analysis (negative-ion mode): 1060.1 (calc. 1060.2, C₄₆H₇₁N₁₄O₁₅).

Analytical HPLC, t_{R}=1.94 min (Gradient 4); t_{R}=1.54 min (Gradient 5).

Tubule Formation Assay.

24 well plates were coated with 30 μl/well of growth factor-reduced (GF-reduced) Matrigel (Becton Dickinson Labware, Bedford, Mass.). Endothelial cells plated on this matrix migrate and differentiate into tubules within 6h of plating as described previously (14). HuDMECs or SVEC 4-10 cells were seeded at a density of 4×10⁴ cells/ml and incubated for 6h in 500 μl of either DMEM+1% FCS alone (control), or this medium±10 ng/ml VEGF or bFGF in the presence or absence of fibrinogen E-fragment, fibrin E-fragment, FpA or α1-24. Assessment of tubule formation involved fixing the cell preparation in 70% ethanol at 4° C. for 15 minutes, rinsing in PBS and staining with haematoxylin and eosin. Three random fields of view in 3 replicate wells for each test condition were visualised under low power (×40 magnification), and colour images captured using a Fuji digital camera linked to a Pentium III computer (containing a frame grabber board). Tubule formation was assessed by counting the number of tubule branches and the total area covered by tubules in each field of view using image analysis software supplied by Scion Image.

Migration Assay

The Boyden chamber technique was adapted from (13) and used to evaluate HuDMEC migration across a porous membrane towards a concentration gradient of either VEGF (10 ng/ml) or bFGF (10 ng/ml). The Neuro Probe 48 well microchemotaxis chamber (Neuro Probe Inc, Cabin John, Md.) was used with 8 μm pore size polycarbonate membranes (Neuro Probe Inc, Cabin John, Md.) coated with 100 μg/ml collagen type IV. 10 ng/ml VEGF or bFGF alone or with various concentrations of fibrinogen E-fragment, fibrin E-fragment, FpA or α1-24 were dissolved in DMEM+1% FCS and placed in the lower wells. The collagen-coated membrane was then placed over this and 5 μl of 25×10⁴ HuDMECs/ml (in DMEM containing 1% FCS) added to the upper chamber. The chambers were then incubated at 37° C. for 4.5h. The chamber was then dismantled, the membrane removed and non-migrated cells scraped off the upper surface. Migrated cells on the lower surface were fixed with methanol, stained with Hema ‘Gurr’ rapid staining kit (Merck, Leics, United Kingdom) and counted using a light microscope (×160 magnification) in 3 random fields per well. Each test condition was carried out in 3-6 replicate wells and each experiment repeated 3 times.

Proliferation Assay

The MTT (3-[4,5-Dimethylthiazol-2-yl]-2,5-diphenyltetrazolium bromide) assay was used as previously described (12) to assess HuDMEC proli0feration induced by VEGF or bFGF in the absence or presence of fibrinogen E-fragment, fibrin E-fragment, FpA or α1-24. HuDMEC were seeded at 3×10³ cells/100 in DMEM+1% FCS±10 ng/ml VEGF or bFGF in test solution into 96 well microtitre plate for 4.5 and 6h. At these time points, a quarter volume of MTT solution (2 mg MTT/ml PBS) was added to each well and each plate was incubated for 4h at 37° C. resulting in an insoluble purple formazan product. The medium was aspirated and the precipitates dissolved in 1001 Σl DMSO buffered at pH 10.5. The absorbance was then read at 540 nm on a Dynex ELISA plate reader.

Cytotoxicity Assay

HuDMECs were seeded at a density of 1-2×10⁵ cells per well in a 24 well-plate in the absence or presence of fibrinogen E-fragment, fibrin E-fragment, FpA or α1-24. After 6h, both live (following removal by trypsinisation) and dead (floating) cells were harvested and cell viability of all cells present assessed using propidium iodide staining of 5000 cells in each of triplicate samples per treatment using a FACScan (Becton Dickinson) equipped with a blue laser excitation of 15 mW at 488 nm. The data was collected and analysed using Cell Quest software (Becton Dickinson).

In vivo Efficacy of Peptides

Experiments were performed on six-week-old Balb/C mice weighing 15 g, obtained from Sheffield Field Laboratories. All experiments were approved by the Home Office Project Licence Number PPL50/1414.

Tumour Cell Culture

The CT26 cell line was maintained by in vitro passage in Dulbecco's Minimal Eagles Medium containing 10% foetal calf serum, and 1% penicillin and streptomycin and maintained at 37° C. in humidified atmosphere of 5% CO₂ in air. The cell line was routinely checked to ensure freedom from mycoplasma (Mycoplasma rapid detection system, Gena-Probe Incorporated, U.S.A.).

Subcutaneous Tumour Implantation

Animals were anaesthetised with an intraperitoneal injection of diazepam (0.5 mg/ml, Dumex Ltd.) and hypnorm (fentanyl citrate 0.0315 mg/ml and fluanisone 1 mg/ml, Janssen Pharmaceutical Ltd.) in the ratio of 1:1 at a volume of 0.1 ml/200 g body weight, with supplementation as required to maintain adequate anaesthesia. Naïve Balb/c mice were immunised s.c into the right flank, following removal of the fur.

Tumour cells were injected at a concentration of 3×10⁵ viable CT26 cells per animal suspended in 100 ul serum free medium. Animals were then allowed to recover.

Tumour growth and animal weights were monitored daily.

Administration of Peptides

Tumour growth was measured daily and when the majority of animals in the cohort had tumour volumes of>100 mm³ but<350 mm³ animals were divided into experimental and control groups. This occurred between 14 and 18 days following implantation of the tumour cell suspension. Animals then received an intraperitoneal (ip) injection of either active drug (peptide α1-24 100 mM; 1001 μl) or vehicle (phophate buffered saline, 100 μl). Daily injections continued until the tumour growth in the control animals reached the maximum burden allowed by Home Office legislation.

Assessment of Tumour Growth

Tumour volumes were assessed by calliper measurements of the perpendicular diameters and volumes estimated using the equation:—
Volume=(a²×b)/2
where a is the smaller and b the larger diameter

Animals were weighed on a daily basis and the general well being monitored.

Statistical Analysis.

All experiments were performed at least three times and data analysed using the Mann-Whitney U test, a non-parametric test that does not assume a Gaussian distribution in the data being analysed. P<0.05 was taken as significant.

### References

1. Folkman J Angiogenesis in cancer, vascular, rheumatoid and other disease. Nature Medicine, 1: 27-31, 1995.
2. Leek R, Harris A L, and Lewis CE Cytokine networks in solid human tumours: regulation of angiogenesis. J. Leuk. Biol., 56: 423-35, 1994.
3. Cao Y Endogenous angiogenesis inhibitors: angiostatin, endostatin, and other proteolytic fragments. Prog Mol Subcell Biol., 20:161-76, 1998.
4. Doolittle R Fibrinogen and Fibrin. Scientific American, 245: 92-101, 1981.
5. Costantini V, Zacharski L R, Memoli V A, Kisiel W, Kudryk B J, and Rousseau S M Fibrinogen deposition without thrombin generation in primary human breast cancer tissue. Cancer Res., 51:349-53, 1991.
6. Dvorak H F, Nagy J A, Feng D, Brown L F, and Dvorak A M Vascular permeability factor/vascular endothelial growth factor and the significance of microvascular hyperpermeability in angiogenesis. Curr Top Microbiol Immunol, 237:97-132, 1999.
7. Thompson W D, Wnag J E H, Wilson S J, and Ganesalinghiam N Angiogenesis and fibrin degradation in human breast cancer. Angiogenesis: Molecular Biology, Clinical Aspects, 245-251, 1994.
8. Thompson W D, Smith E B, Stirk C M, Marshall F I, Stout A J, and Kocchar A Angiogenic activity of fibrin degradation products is located in fibrin fragment E. J. Pathol, 168: 47-53, 1992.
9. Malinda K M, Ponce L, Kleinman H K, Shackelton L M, and Millis A J Gp38k, a protein synthesized by vascular smooth muscle cells, stimulates directional migration of human umbilical vein endothelial cells. Exp Cell Res 250:168-73, 1999.
10. Shen J, Ham R G, Karmiol S Expression of adhesion molecules in cultured human pulmonary microvascular endothelial cells. Microvasc Res., 50:360-72, 1995.
11. Liu J. Kolath J. Anderson J. Kolar C, Lawson TA, Talmadge J. and Gmeiner W H Positive interaction between 5-FU and FdUMP[10] in the inhibition of human colorectal tumour cell proliferation. Antisense Nucleic Acid Drug Dev., 9(5):481-6, 1999.
12. Dejano E, Languino L R, Polentarutti N, Balconi G, Ryckewaert J J, Larrieu M J, Donati M B, Mantovani A, and Marguerie G Interaction between fibrinogen and cultured endothelial cells. J. Clin. Invest., 75: 11-18, 1985.
13. Bootle-Wilbraham C A, Tazzyman S, Marshall J M, Lewis C E. Fibrinogen E-fragment inhibits the migration and tubule formation of human dermal microvascular endothelial cells in vitro. Cancer Research (2000) 60: 4719-4724
14. Marsh H C, Meinwald Y C, Lee S, Martinelli R A, Scheraga H A. Mechanism of action of thrombin on fibrinogen: NMR evidence for a beta-bend at or near fibrinogen A alpha Gly(P5)-Gly(P4). Biochemistry (1985) 24: 2806-2812.
15. Gellman S. H., Acc. Chem. Res. (1998)31: 173-180.

1. A polypeptide of 15 or less amino acid residues comprising a sequence selected from the group consisting of:
X₁FLAEX₆X₇X₈V
DX₂LAEX₆X₇X₈V
DFX₃AEX₆X₇X₈V
DFLX₄EX₆X₇X₈V
DFLAX₅X₆X₇X₈V
DFLAEX₆X₇X₈V
DFLAEX₆X₇X₈X₉
wherein X is any proteinogenic or non-proteinogenic amino acid residue and which polypeptide has anti-angiogenic activity.

2. A polypeptide according to claim 1 wherein one or more amino acids are replaced by a conservative proteinogenic or non-proteinogenic amino acid.

3. A polypeptide according to claim 1 wherein X₁ is an acidic amino acid or wherein X₂, X₃, X₄, X₅ are selected from the groups consisting of glycine or alanine, valine, leucine, isoleucine, proline, wherein X₆, X₇, X₈ any amino acid and wherein X₉ is a non-polar amino acid.

4. A polypeptide according to claim 1 wherein X₁ is asparatic acid, X₂ is phenylalanine, X₃ is leucine, X₄ is alanine, X₅ is glutamic acid, X₆, X₇, and X₈ are glycine, and X₉ is valine.

5. A polypeptide according to the previous claims wherein one or more of amino acid residues X₁ and X₅ are replaced by an acidic non-proteinogenic amino acid, preferably selected from the group consisting of: gamma-carboxy-L-glutamic acid, L-2-aminoadipic acid, L-3-aminoadipic acid and (+/−)-1minocyclopentane-cis-1,3-dicarboxylic acid.

6. A polypeptide according to the previous-claims wherein X₂ is selected from the group consisting of L-2-naphthylalanine, L-1,2,3,4-tetrahydroisoquinoline-3-carboxylic acid and 4,4′-biphenylalanine.

7. A polypeptide according to the previous claims wherein one or more of amino acid residues X₃, X₄ or X₉ are replaced by a hydrophobic non-proteinogenic amino acid, preferably selected from the group consisting of 1-aminocyclopropanecarboxylic acid, 3-aminopentane-3-carboxylic acid, R-2-amino-2-cyclohexyl-propanoic acid.

8. A polypeptide according to the previous claims comprising a sequence selected from the group consisting of:
GX₁X₂X₃X₄X₅X₆X₇X₈X₉
EGX₁X₂X₃X₄X₅X₆X₇X₈X₉
GEGX₁X₂X₃X₄X₅X₆X₇X₈X₉
wherein X₁, X₂, X₃, X₄, X₅, X₆, X₇, X₈ and X₉, respectively, are defined as above.

9. A polypeptide according to the previous claims comprising a sequence selected from the group consisting of:
X₁X₂X₃X₄X₅X₆X₇X₈X₉R
X₁X₂X₃X₄X₅X₆X₇X₈X₉RG
X₁X₂X₃X₄X₅X₆X₇X₈X₉RGP
wherein X₁, X₂, X₃, X₄, X₅, X₆, X₇, X₈ and X₉, respectively, are defined as above.

10. A polypeptide according to the previous claim wherein one or more of residues X₇X₈X₉ are replaced by a flexible chemical linker.

11. A polypeptide according to claim 10 wherein said linker has a backbone of comprises 3 to 40 atoms.

12. The polypeptide of the preceding claim wherein said polypeptide has a length of 9, 10, 11, 12, 13, or 14 amino acid residues.

13. A polypeptide according to claim 9 which polypeptide comprises an amino acid sequence of the sequence XₐFLAEGGGVX_{b}G, wherein X is any amino acid.

14. The polypeptide of claim 13 wherein Xₐ is an acidic amino acid and X_{b} is a basic amino acid.

15. The polypeptide of claim 14 wherein X_{b} is selected from the group consisting of L-2,4-diaminobutyric acid, R-aminocamitine, L-alpha-amino-gamma-guanidinobutyric acid and omithine.

16. A polypeptide selected from the group consisting of:
i) a peptide of the sequence,
XXXXXLXEXXGXXXPRVXXR,
or part thereof, wherein X is any amino acid residue;
ii) a peptide as represented in (i) wherein amino acid residue X is selected from the following group: glycine, alanine, valine, leucine, isoleucine, proline; and
iii) a peptide represented in (i) or (ii) which has anti-angiogenic activity.

17. A polypeptide according to any of claim 16 which polypeptide consists of an amino acid sequence selected from the group consisting of:
GEG DFL AEG GGV RGP RVVE R
GEG DFL AEG GGX RGP RVVE R
GEG DFL AEG GGV XGP RVVE R
GEG DFL AEG GGV RXP RVVE R
GEG DFL AEG GGV RGP RVXE R
GEG DFL AEG GGV RGP RVVXR
GEG DFL AEG GGXXXP RVVX R
GEG DFL AEG GGXXXP RVXXR.
wherein X is any amino acid residue.

18. A polypeptide according to claim 16 wherein X is an amino acid residue selected from the group consisting of glycine, alanine, valine, leucine, isoleucine, or proline.

19. A polypeptide according to any of the previous claims which polypeptide is modified at the N-terminus.

20. A polypeptide according to claim 19 wherein said modification is selected from the group consisting of carboxamide, carbamate, urea, sulphonamide, acetylation and alkylation.

21. A polypeptide according to the previous claims which polypeptide is modified at the C-terminus.

22. A polypeptide according to claim 21 wherein said modification is selected from the group consisting of amidate, carboxylic acid, ester, carboxamide and alcohol.

23. A polypeptide according to the previous claims-which polypeptide is modified at the N-terminus and at the C-terminus.

24. A polypeptide according to the previous claims which polypeptide has one or more L-amino acid replaced by the corresponding D-amino acid.

25. A polypeptide according to the previous claims which polypeptide has one or more amino-acids are replaced by an α-methyl derivative and/or an N-methyl derivative.

26. A polypeptide according to the previous claim wherein said polypeptide is modified by cyclisation.

27. A polypeptide according to claim 26 wherein said cyclisation is a head to tail cyclisation, side-chain to side-chain cyclisation, side-chain to end cyclisation, branched cyclisation or backbone to backbone cyclisation.

28. A polypeptide according to the previous claims wherein said polypeptide comprises one or more chemical modifications of the backbone.

29. A polypeptide according to claim 28 wherein said modified backbone is selected from the group consisting of: beta-peptide backbone, depsipeptide backbone, oligosulfonamide backbone, oliogurea and thiourea backbone, oligocarbamate backbone, peptoid backbone and azapeptide backbone.

30. The polypeptide of the preceding claims-wherein said polypeptide consists of said sequence.

31. The polypeptide of the preceding claims wherein said polypeptide inhibits cell proliferation by at least 10 percent relative to a control without said polypeptide in a proliferation assay.

32. A polypeptide according to claim wherein said polypeptide inhibits tumour growth by at least 10 percent relative to a control without said polypeptide.

33. A pharmaceutical composition comprising a polypeptide according to claim 1.

34. A pharmaceutical composition comprising two or more polypeptides according to claim 1 wherein said agent has anti-angiogenic activity.

35. A pharmaceutical composition according to claim 34 wherein said two or more polypeptides are linked by a linker molecule.

36. A pharmaceutical composition according to claim 34 wherein said pharmaceutical composition comprises a plurality of polypeptides.

37. A pharmaceutical composition according to claim 34 wherein said pharmaceutical composition comprises 3, 4, 5, 6, 7, 8, 9, or 10 polypeptides linked together as an oligomeric polypeptide.

38. A pharmaceutical composition according to claim 34 wherein said pharmaceutical composition is a dimer of two polypeptides.

39. Use of a polypeptide according to claim 1 for the manufacture of a medicament for use in the treatment of cancer.

40. An isolated nucleic acid molecule consisting of a DNA sequence encoding a polypeptide according to claim 1.

41. An isolated nucleic acid molecule which anneals under stringent hybridisation conditions to the sequence according to claim 40.

42. A vector comprising a nucleic acid molecule according to claim 40 operably linked to a promoter.

43. A vector according to claim 42 which is an expression vector adapted for prokaryotic or eukaryotic cell expression.

44. A cell transformed/transfected with the nucleic acid according to claim 40 or the vector according to claim 42.

45. A method for the production of polypeptides according to claim 1:
i) providing a cell according to claim 44;
ii) providing conditions conducive to the manufacture of said polypeptides; and
iii) purifying said polypeptides from a cell, or a cells culture environment.

46. A non-human, transgenic animal characterised in that said animal incorporates a nucleic acid molecule encoding a polypeptide according to of claim 1.

47. A method to treat an animal which would benefit from inhibition of angiogenesis comprising:
i) administering an effective amount of a polypeptide according to claim 1 to an animal to be treated;
ii). monitoring the effects of said polypeptide on the inhibition of angiogenesis.

48. A method to treat an animal which would benefit from inhibition of angiogenesis comprising:
i) administering an effective amount of an pharmaceutical composition according to claim 33 to an animal to be treated;
ii). monitoring the effects of said agent on the inhibition of angiogenesis.

49. A method to treat an animal which would benefit from inhibition of angiogenesis comprising:
i) administering an effective amount of a nucleic acid molecule according to claim 40 or a vector according to claim 42 to an animal to be treated;
ii). monitoring the effects of said nucleic acid or vector on the inhibition of angiogenesis.

50. An imaging agent comprising a polypeptide according to any of claim 1.
